(19) United States
(12) Patent Application Publication
(10) Pub. No.: US 2008/0182865 A1
(43) Pub. Date: Jul. 31, 2008

(54) HISTONE DEACETYLASE INHIBITORS SENSITIZE CANCER CELLS TO EPIDERMAL GROWTH FACTOR INHIBITORS

(76) Inventors: Samir E. Witta, Greenwood Village, CO (US); Paul A. Bunn, Steamboat Springs, CO (US); Harry A. Drabkin, Charleston, SC (US); Robert M. Gemmill, Charleston, SC (US); Daniel Chuen-Fong Chan, Denver, CO (US)

Correspondence Address: SHERIDAN ROSS PC, 1560 BROADWAY, SUITE 1200, DENVER, CO, 80202, US

(21) Appl. No.: 11/861,033

(22) Filed: Sep. 25, 2007

Related U.S. Application Data
(63) Continuation-in-part of application No. 11/908,388.
(60) Provisional application No. 60/951,445, filed on Jul. 23, 2007.
(60) Provisional application No. 60/660,893, filed on Mar. 11, 2005.
(60) Provisional application No. 60/951,445, filed on Jul. 23, 2007.

(51) Int. Cl. A61K 31/498; A61K 31/4406

(52) U.S. Cl. 514/266.24; 514/357

(57) ABSTRACT

Disclosed is the use of a combination of histone deacetylase inhibitors and kinase inhibitors with anti-EGFR activity.

### CROSS-REFERENCE TO RELATED APPLICATIONS

This application claims the benefit of priority under 35 U.S.C. § 119(e) from U.S. Provisional Application Ser. No. 60/951,445, filed Jul. 23, 2007. This application is also a continuation-in-part of U.S. patent application Ser. No. 11/908,388, filed Sep. 11, 2007, which is a national stage application under 35 U.S.C. 371 of PCT Application No. PCT/US2006/009078, filed Mar. 13, 2006, which claims the benefit of priority under 35 U.S.C. § 119(e) from U.S. Provisional Application Ser. No. 60/660,893, filed Mar. 11, 2005. The entire disclosure of each of U.S. Provisional Application Ser. No. 60/951,445, U.S. patent application Ser. No. 11/908,388, PCT Application No. PCT/US2006/009078, and U.S. Provisional Application Ser. No. 60/660,893, is incorporated herein by reference.

### FIELD OF THE INVENTION

This application generally relates to the use of a combination of histone deacetylase inhibitors and epidermal growth factor receptor (EGFR) inhibitors to treat cancer.

### BACKGROUND OF THE INVENTION

Non-small cell lung cancer (NSCLC) is the leading cause of cancer death in the world. While chemotherapy has produced modest survival benefits in advanced stages, standard two-drug combinations generate considerable toxicity and require intravenous administration. Progress in the field of lung cancer biology led to the development of small molecule inhibitors of target proteins involved in the proliferation, apoptosis and angiogenesis. Targeted therapy agents such as imatinib and trastuzumab produced consistent survival benefit in chronic myeloid leukemia, gastrointestinal stromal tumors (GIST) and breast cancers that overexpress the target proteins. The epidermal growth factor receptor (EGFR) superfamily, including the four distinct receptors EGFR/erbB-1, HER2/erbB-2, HER3/erbB-3, and HER4/erbB-4, was early identified as a potential therapeutic target in solid tumors. After ligand binding, these receptors homo- and heterodimerize, and the tyrosine-kinase domain is activated, initiating a cascade of events implicated in the development and progression of cancer through effects on cell-cycle progression, apoptosis, angiogenesis, and metastasis. EGFR is overexpressed in many human epithelial malignancies, including NSCLC.

Given the biological importance of the EGFR molecular network in carcinomas, several molecules were synthesized to inhibit the tyrosine kinase domain of EGFR. Among the most promising of these new drugs are gefitinib (ZD 1839, IRESSA®, AstraZeneca, UK), and erlotinib (OSI 774, TARCEVA®, Genentech, USA). Both are orally active, selective EGFR tyrosine-kinase inhibitors (EGFR-TKI) that demonstrated antitumor activity against a variety of human cancer cell lines expressing EGFR. Likewise, both have well documented activity as single agents in phase I studies including chemotherapy resistant NSCLC patients who had response rates of about 10%. Activity was confirmed in large phase II trials showing response rates of 19-26% in previously untreated, advanced NSCLC patients, and 12-18% in patients who had failed one or more prior chemotherapy combinations. More recently, a survival benefit with erlotinib as a second or third line therapy was reported in a trial performed by the National Cancer Institute Canada. Other drugs with EGFR TKI activity are in clinical use or clinical development such as Lapatinib (GW572016) and Vandetanib (ZACTIMA™; ZD6474).

In phase II trials with gefitinib, no correlation was detected between EGFR protein expression and response to therapy. Some patients with squamous cell carcinomas have lower response rates compared to patients with adenocarcinoma despite their higher rates of EGFR expression. Recent reports showed that specific missense and deletion mutations in the tyrosine kinase domain of the EGFR gene are significantly associated with gefitinib sensitivity. However, while objective response has been reported in up to 18% and symptomatic improvement in 40% of the unselected gefitinib treated NSCLC patients, the low frequency of these mutations in unselected US patients suggest that other mechanisms are also involved in the response to gefitinib. EGFR interacts with cell adhesion molecules including the integrins and E-cadherin (E-cad, CDH1). E-cad is a calcium-dependent epithelial cell adhesion molecule that plays an important role in tumor invasiveness and metastatic potential. Reduced E-cad expression is associated with tumor cell dedifferentiation, advanced stage and reduced survival in patients with NSCLC. E-cad-mediated cell adhesion requires intracellular attachment to the actin cytoskeleton through the interaction with β-, α- and γ-catenin. Activation of EGFR leads to a loss of the membranous localization and proteosomal degradation of E-cad and β-catenin. E-cad is also involved in regulation of EGFR and its downstream targets. E-cad inhibits ligand-dependent activation of EGFR and other RTKs. On the other hand, E-cad action on neighboring cells leads to PI 3-kinase-dependent activation of AKT and the rapid translocation of AKT to the nucleus. E-cad also stimulates the MAPK pathway through the ligand-independent activation of EGFR. At the transcriptional level, E-cad expression is regulated by the wnt/β-catenin signaling, the EGFR signaling via ERK or caveolin, the transcription factor AP-2, the basis helix-loop-helix E12/E47 factor, and by several zinc finger transcription factors including the Slug/Snail family, SIP1 and TF8 (ZEB-1, ZFHX1A, AREB6, δEF1). These zinc-finger transcription factors regulate the expression of several genes via the interaction with two 5′-CACCTG (E-box) promoter sequences. This regulation is facilitated by the interaction with CtBP, which recruits histone deacetylases (HDAC) leading to chromatin condensation and gene silencing. Inhibiting HDAC using trichostatin A (TSA) in lung cancer cell lines led to the activation of E-cad.

To date, several mammalian HDACs have been identified and grouped into 3 classes (Class I-III). HDAC inhibitors are an emerging class of therapeutic agents that promote differentiation and apoptosis in hematologic and solid malignancies through chromatin remodeling and gene expression regulation. Several HDAC inhibitors were identified including benzamides (MS-275), short-chain fatty acids (i.e., Sodium phenylbutyrate); hydroxamic acids (i.e., suberoylanilide hydroxamic acid and trichostatin A); cyclic tetrapeptides containing a 2-amino-8-oxo-9,10-epoxy-decanoyl moiety (i.e., trapoxin A) and cyclic peptides without the 2-amino-8-oxo-9,10-epoxy-decanoyl moiety (i.e., FK228). The majority of these are undergoing clinical trials. MS-275 (Schering AG) is a benzamide HDAC inhibitor undergoing Phase I investigation in hematologic and solid malignancies. MS-275 is rapidly absorbed and has a half-life of 100 hours; changes in histone acetylation have persisted for several weeks following the administration of MS-275.

It is of great interest to identify patients that would benefit from EGFR inhibitors and to identify treatments that can improve the responsiveness of cancer cells which are resistant to EGFR inhibitors, particularly for use in cancer cells that express EGFR. In particular, it would be desirable to find treatment regimens that would increase the sensitivity of a cancer cell line that expresses EGFR to EGFR inhibitors.

### SUMMARY OF THE INVENTION

One embodiment of the present invention relates to a method to treat a patient with cancer. The method includes the step of administering to the patient a combination of at least one histone deacetylase (HDAC) inhibitor and at least one epidermal growth factor receptor (EGFR) inhibitor.

One embodiment of the invention relates to a method to treat a patient with cancer. The method includes the step of administering to the patient a combination of at least one selective histone deacetylase (HDAC) inhibitor and at least one kinase inhibitor with anti-EGFR activity.

In one embodiment, the HDAC inhibitor, including the selective HDAC inhibitor, improves the sensitivity of the cancer to the kinase inhibitor.

In one embodiment, suitable dosages are total daily dosage of the HDAC inhibitor are between about 25 to 4000 mg/m². They can be administered in various cycles: once daily at a dose of about 200 to 600 mg; twice daily at a dose of about 200 to 400 mg; twice daily at a dose of about 200 to 400 mg intermittently (e.g. three, four, or five days per week); three times daily at a dose of about 100 to 250 mg; daily dose is 200 mg, which can be administered once-daily, twice-daily, or three-times daily; daily dose is 300 mg, which can be administered once-daily or twice-daily; daily dose is 400 mg, which can be administered once-daily or twice-daily.

In one embodiment, suitable dosages are total daily dosage of the EGFR inhibitor are between about 25 to 4000 mg/m². They can be administered in various cycles: once daily at a dose of about 200 to 600 mg; twice daily at a dose of about 200 to 400 mg; twice daily at a dose of about 200 to 400 mg intermittently (e.g. three, four, or five days per week); three times daily at a dose of about 100 to 250 mg; daily dose is 200 mg, which can be administered once-daily, twice-daily, or three-times daily; daily dose is 300 mg, which can be administered once-daily or twice-daily; daily dose is 400 mg, which can be administered once-daily or twice-daily.

In one embodiment, the EGFR and/or HDAC inhibitor is administered systemically to attain a blood level from about 0.01 μM to about 10 μM. In additional or further embodiments, the therapeutic composition is administered at a sufficient dosage to attain a blood level of from about 0.05 μM to about 10 μM. In additional or further embodiments, the blood level of is from about 0.1 μM to about 7 μM.

In one embodiment, the HDAC inhibitor described herein is administered with an EGFR inhibitor. The co-administered compounds can be administered in a variety of cycles: the HDAC inhibitor can be administered continuously, daily, every other day, every third day, once a week, twice a week, three times a week, bi-weekly, or monthly, while the EGFR inhibitor is administered continuously, daily, one day a week, two days a week, three days a week, four days a week, five days a week, six days a week, bi-weekly, or monthly. In one aspect, the HDAC inhibitor is administered prior to (before) the EGFR inhibitor is administered. The HDAC inhibitor and the EGFR inhibitor can be administered in, but are not limited to, any combination of the aforementioned cycles. In one non-limiting example, the HDAC inhibitor is administered three times a week for the first two weeks followed by no administration for four weeks, and the EGFR inhibitor is administered continuously over the same six week period. In yet another non-limiting example, the HDAC inhibitor is administered once a week for six weeks, and the EGFR inhibitor is administered every other day over the same six week period. In yet another non-limiting example, the HDAC inhibitor is administered the first two days of a week, and the EGFR inhibitor is administered continuously for all seven days of the same week.

In one aspect, the HDAC inhibitor is a selective HDAC inhibitor and the EGFR inhibitor is lapatinib. In one aspect, such an HDAC inhibitor is MS-275.

In one aspect, the HDAC inhibitor is MS-275 and the EGFR inhibitor is gefitinib. In this aspect, the dosing regime can include administration of MS-275 at 2 mg/m² orally weekly for 4 weeks followed by administration of gefitinib at 250 mg orally per day for 4 weeks. In one aspect, the HDAC inhibitor is administered orally 1-4 weeks followed by administration of an EGFR TKI for 1-4 weeks.

In another aspect, the combination is administered over substantially the same time period. For example, in this aspect, the dosing regime can include administration of MS-275 at 2 mg/m² orally weekly for 4 weeks coadministered with gefitinib at 250 mg orally per day for 4 weeks. In another aspect, the dosing regime can include administration of MS-275 orally weekly for 1-4 weeks coadministered with an EGFR TKI for 1-4 weeks. In another aspect, the dosing regime can include administration of MS-275 orally weekly for 4 weeks coadministered with an EGFR TKI orally per day for 4 weeks.

In addition to the administration of the compounds in cycles, the cycles themselves may consist of varying schedules. In one embodiment, a cycle is administered weekly. In additional embodiments, a cycle is administered for one week with one, two, three, four, six, or eight weeks off before repeating the cycle. In further embodiments, a cycle is administered for two weeks with one, two, three, four, six, or eight weeks off before repeating the cycle. In still further embodiments, the cycle is administered for three, four, five, or six weeks, with one, two, three, four, six, or eight weeks off before repeating the cycle.

When the HDAC inhibitor plus EGFR inhibitor combination is administered with an additional treatment such as radiotherapy, the radiotherapy can be administered at 1 day, 2 days, 3 days, 4 days, 5 days, 6 days, 7 days, 14 days, 21 days, or 28 days after administration of at least one cycle of a compound. In additional embodiments, the radiotherapy can be administered in any variation of timing with any variation of the aforementioned cycles for a compound. Additional schedules for co-administration of radiotherapy with cycles of a compound will be known in the art, can be further determined by appropriate testing, clinical trials, or can be determined by qualified medical professionals.

The HDAC inhibitor plus EGFR inhibitor combinations described herein can also be administered in combination with at least one second chemotherapeutic compound (e.g. pharmaceuticals, small-molecule compounds, antibodies and fragments thereof, immune system modulating proteins, antibiotics, or other biologic therapy), radiotherapy, or surgery. Such co-administration is believed to increase efficacy, provide synergistic effect, and/or provide increased therapeutic value to each agent, compound, or additional treatment (e.g. radiotherapy or surgery).

When the HDAC inhibitor plus EGFR inhibitor combination is administered with an additional treatment such as surgery, the compound is administered 1, 2, 3, 4, 5, 6, 7, 14, 21, or 28 days prior to surgery. In additional embodiments, at least one cycle of the compound is administered 1, 2, 3, 4, 5, 6, 7, 14, 21, or 28 days after surgery. Additional variations of administering compound cycles in anticipation of surgery, or after the occurrence of surgery, will be known in the art, can be further determined by appropriate testing and/or clinical trials, or can be determined by assessment of qualified medical professionals.

In addition to the aforementioned examples and embodiments of dosages, cycles, and schedules of cycles, numerous permutations of the aforementioned dosages, cycles, and schedules of cycles for the co-administration of a compound with a second chemotherapeutic compound, radiotherapy, or surgery are contemplated herein and can be administered according to the patient, type of cancer, and/or appropriate treatment schedule as determined by qualified medical professionals.

In one aspect, the combination is administered sequentially. For example, in this aspect, at least a substantial portion of the HDAC inhibitor can be administered before a substantial portion of the EGFR inhibitor is administered.

Another embodiment of the present invention relates to a method to treat a patient with an epidermal growth factor receptor (EGFR) inhibitor-resistant cancer by sensitizing the cancer cells to EGFR inhibitors. The method includes administering to the patient a combination of at least one histone deacetylase (HDAC) inhibitor and at least one EGFR inhibitor. In one aspect of this embodiment, the method additionally comprises the step of evaluating the cancer to predict resistance to an EGFR inhibitor prior to administration of the therapeutic composition. For example, the step of evaluating the cancer can include: (a) detecting in a sample of tumor cells from a patient a level of a biomarker selected from: (i) a level of amplification of the epidermal growth factor receptor (EGFR) gene; (ii) a level of polysomy of the EGFR gene; (iii) a level of amplification of the human tyrosine kinase receptor-type receptor (HER2) gene; and (iv) a level of polysomy of the HER2 gene; (b) comparing the level of the biomarker in the tumor cell sample to a control level of the biomarker selected from: (i) a control level of the biomarker that has been correlated with sensitivity to the EGFR inhibitor; and (ii) a control level of the biomarker that has been correlated with resistance to the EGFR inhibitor; and (c) selecting the patient as being predicted to not benefit from therapeutic administration of the EGFR inhibitor, or being predicted to benefit from the combination of HDAC inhibitor and EGFR inhibitor, if the level of the biomarker in the patient's tumor cells is statistically less than the control level of the biomarker that has been correlated with sensitivity to the EGFR inhibitor, or if the level of the biomarker in the patient's tumor cells is statistically similar to or less than the level of the biomarker that has been correlated with resistance to the EGFR inhibitor.

In another aspect of this embodiment, the method additionally comprises the steps of: (a) detecting a level of expression of epidermal growth factor receptor (EGFR) protein in the tumor cell sample; (b) comparing the level of EGFR protein expression in the tumor cell sample to a control level of EGFR protein expression selected from: (i) a control level that has been correlated with sensitivity to the EGFR inhibitor; and (ii) a control level that has been correlated with resistance to the EGFR inhibitor; and (c) selecting the patient as being predicted to not benefit from therapeutic administration of the EGFR inhibitor, or being predicted to benefit from the combination of HDAC inhibitor and EGFR inhibitor, if the level of EGFR protein expression in the patient's tumor cells is statistically less than the control level of EGFR protein expression that has been correlated with sensitivity to the EGFR inhibitor, or if the level of EGFR protein expression in the patient's tumor cells is statistically similar to or less than the level of EGFR protein expression that has been correlated with resistance to the EGFR inhibitor.

In a further aspect of this embodiment, the method includes the additional steps of: (d) detecting in the sample of tumor cells a level of expression of the E-cadherin protein; (e) comparing the level of E-cadherin expression in the tumor cell sample to a control level of E-cadherin expression selected from: (i) a control level that has been correlated with sensitivity to an EGFR inhibitor; and (ii) a control level that has been correlated with resistance to an EGFR inhibitor; and (f) selecting the patient as being predicted to benefit from the combination of HDAC inhibitor and EGFR inhibitor, if the level of E-cadherin expression in the patient's tumor cells is statistically reduced compared to the control level of E-cadherin expression that has been correlated with sensitivity to an EGFR inhibitor, or if the level of E-cadherin expression in the patient's tumor cells is statistically similar than the level of E-cadherin expression that has been correlated with resistance to an EGFR inhibitor.

In another further aspect of this embodiment, the method includes the additional steps of: (d) detecting in the sample of tumor cells a level of expression of at least one component of TF8; (e) comparing the level of expression of at least one component of TF8 in the tumor cell sample to a control level of expression of at least one component of TF8 selected from: (i) a control level that has been correlated with sensitivity to an EGFR inhibitor; and (ii) a control level that has been correlated with resistance to an EGFR inhibitor; and (f) selecting the patient as being predicted to benefit from the combination of HDAC inhibitor and EGFR inhibitor, if the level of expression of at least one component of TF8 in the patient's tumor cells is statistically increased compared to the control level of expression of at least one component of TF8 that has been correlated with sensitivity to an EGFR inhibitor, or if the level of expression of at least one component of TF8 in the patient's tumor cells is statistically similar than the level of expression of at least one component of TF8 that has been correlated with resistance to an EGFR inhibitor.

Yet another embodiment of the invention relates to a method to treat a patient with a cancer that is resistant to at least one epidermal growth factor receptor (EGFR) inhibitor, comprising administering to the patient a combination of at least one histone deacetylase (HDAC) inhibitor and at least one epidermal growth factor receptor (EGFR) inhibitor, wherein the cancer is an epithelial malignancy.

In any of the embodiments and aspects of the present invention, the HDAC inhibitor can include, but is not limited to, a hydroxamic acid, a carboxylic acid, a benzamide, an epoxide, a short-chain fatty acid, a cyclic tetrapeptide containing a 2-amino-8-oxo-9,10-epoxy-decanoyl moiety, and a cyclic peptide without the 2-amino-8-oxo-9,10-epoxy-decanoyl moiety. A hydroxamic acid can include, but is not limited to, suberoylanilidine hydroxamic acid, TSA, and SAHA. A carboxylic acid can include, but is not limited to, butanoic acid, valproic acid, and 4-phenylbutanoic acid. A benzamide can include, but is not limited to, N-acetyldinaline and MS-275. An epoxide can include, but is not limited to, trapoxin, depeudecin, and depsipeptide FK 228. In a preferred embodiment, the HDAC inhibitor is MS-275. In one aspect, MS-275 is administered in a dosing regime comprising administering MS-275 at 2 mg/m² orally weekly for 4 weeks or at 4 mg/m² orally biweekly for 4 weeks. In one aspect, the HDAC inhibitor can include, but is not limited to, Savicol®, Baceca®, MGCD0103, MG98 (an antisense molecule), and LBH589. In one aspect, the HDAC inhibitor is a selective HDAC inhibitor. In one aspect, the selective HDAC inhibitor is selected from MS-275 or MGCD0103.

In any of the embodiments or aspects of the present invention, the EGFR inhibitor includes, but is not limited to, a kinase inhibitor with anti-EGFR activity. In one aspect, the kinase inhibitor is a dual kinase inhibitor. Such an EGFR inhibitor can include a variety of tyrosine kinase inhibitors, including, but not limited to, anti-EGFR family tyrosine kinase inhibitors (small molecules) and anti-EGFR monoclonal antibodies. In one aspect, the EGFR inhibitor includes, but is not limited to, EGFR-specific and irreversible inhibitors. The EGFR inhibitor can include, but is not limited to, gefitinib, erlotinib, lapatinib, vandetanib, sorafenib, sutent, PKI-166, a PAN-HER (human EGF receptor family) reversible inhibitor, a PAN-HER irreversible inhibitor, an agonist of sorafenib, an agonist of sutent, an agonist of gefitinib, an agonist of lapatinib, an agonist of vandetanib, and an agonist of erlotinib. In a preferred embodiment, the EGFR inhibitor is gefitinib, lapatinib, vandetanib, sutent, sorafenib or erlotinib. Gefitinib can be administered, for example, in a dosing regime comprising administration of 250 mg PO per day. Erlotinib can be administered, for example, in a dosing regime comprising administration of 150 mg PO per day. Lapatinib can be administered, for example, in a dosing regime comprising administration of 1,250 mg (5×250 mg tablets) PO per day in a cycle of 21 days. Vandetanib can be administered, for example, in a dosing regime comprising administration of 300 mg PO per day. Sorafenib can be administered, for example, in a dosing regime comprising administration of 400 mg taken twice PO per day. Sutent can be administered, for example, in a dosing regime comprising administration of 50 mg PO per day. In some embodiments, Sutent is administered in a cycle of four week use followed by a two week stop.

In any of the above-described embodiments or aspects of the invention, the cancer can include, but is not limited to, an epithelial malignancy, a cancer of epithelial origin, a lung cancer (e.g., a non-small cell lung cancer), a breast cancer, a skin cancer, a bladder cancer, a colon cancer, a gastro-intestinal (GI) cancer, a prostate cancer, a pancreatic cancer, a uterine cancer, a cervical cancer, a ovarian cancer, a esophageal cancer, a stomach cancer, or a laryngeal cancer. In one aspect, the cancer is resistant to EGFR inhibitors. For example, in one aspect, the cancer comprises cancerous cells having low or no gain in copy number of the EGFR gene or low or no gain in copy number of the HER2 gene, or a combination thereof, as compared to cancerous cells that are sensitive to EGFR inhibitors. In one aspect, the cancer comprises cancerous cells having reduced expression of EGFR protein as compared to cancerous cells that are sensitive to EGFR inhibitors. In one aspect, the cancer comprises cancerous cells having a reduced level of E-cadherin gene expression as compared to cancerous cells that are sensitive to EGFR inhibitors. In one aspect, the cancer comprises cancerous cells having an enhanced level of at least one component of TF8 expression as compared to cancerous cells that are sensitive to EGFR inhibitors. Such a component can include ZEB1.

Another embodiment of the present invention relates to a method to select a cancer patient who is predicted to benefit from therapeutic administration of a combination of at least one histone deacetylase (HDAC) inhibitor and at least one epidermal growth factor receptor (EGFR) inhibitor. The method includes the steps of: (a) detecting in the sample of tumor cells a level of expression of the E-cadherin protein; (b) comparing the level of E-cadherin expression in the tumor cell sample to a control level of E-cadherin expression selected from: (i) a control level that has been correlated with sensitivity to an EGFR inhibitor; and (ii) a control level that has been correlated with resistance to an EGFR inhibitor; and (c) selecting the patient as being predicted to benefit from the combination of HDAC inhibitor and EGFR inhibitor, if the level of E-cadherin expression in the patient's tumor cells is statistically reduced compared to the control level of E-cadherin expression that has been correlated with sensitivity to an EGFR inhibitor, or if the level of E-cadherin expression in the patient's tumor cells is statistically similar than the level of E-cadherin expression that has been correlated with resistance to an EGFR inhibitor.

Another embodiment of the present invention relates to a method to select a cancer patient who is predicted to benefit from therapeutic administration of a combination of at least one histone deacetylase (HDAC) inhibitor and at least one epidermal growth factor receptor (EGFR) inhibitor. The method includes the steps of: (a) detecting in the sample of tumor cells a level of amplification of zinc finger transcription factor genes; (b) comparing the level of amplification of zinc finger transcription factor genes in the tumor cell sample to a control level of amplification of zinc finger transcription factor genes selected from: (i) a control level that has been correlated with sensitivity to an EGFR inhibitor; and (ii) a control level that has been correlated with resistance to an EGFR inhibitor; and (c) selecting the patient as being predicted to benefit from the combination of HDAC inhibitor and EGFR inhibitor, if the level of amplification of zinc finger transcription factor genes in the patient's tumor cells is statistically greater compared to the control level of amplification of zinc finger transcription factor genes that has been correlated with sensitivity to EGFR inhibitors, or if the level of amplification of zinc finger transcription factor genes in the patient's tumor cells is statistically similar than the level of amplification of zinc finger transcription factor genes that has been correlated with resistance to EGFR inhibitors.

### BRIEF DESCRIPTION OF THE FIGURES OF THE INVENTION

FIG. 1A is schematic drawing showing the general structure of HDAC inhibitors.

FIG. 1B shows examples of HDAC inhibitory chemicals. TSA(1) and SAHA(2) are hydroxamic acids; butanoic acid(3), valproic acid(4) and 4-phenylbutanoic acid(5) are carboxylic acids; MS-275(6) and N-acetyldinaline(7) are benzamides; depeudecin(8) and trapoxine A(9) are epoxides; also shown are apicidin(10) and depsipeptide FK228(11).

FIG. 2 is a graph showing the effect of treatment with gefitinib alone or a combination of gefitinib and MS-275 on H157 cells.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention generally relates to a method to treat a patient with cancer, and particularly a cancer that expresses epidermal growth factor receptor (EGFR) and is resistant to EGFR inhibitors, such as, but not limited to, gefitinib. The present inventors have discovered that EGFR resistant cancers such as EGFR resistant non-small cell lung cancer (NSCL), bladder cancer and colorectal cancer (CRC) have greater responsiveness rates to EGFR therapy when pre-treated or co-treated with a histone deacetylase inhibitor. The method generally includes administering to such patient a combination type therapy comprising a histone deacetylase inhibitor and an EGFR inhibitor. In one embodiment, the histone deacetylase inhibitor and the EGFR inhibitor are administered in sequential order. The method also includes evaluating a patient's cancer for sensitivity or resistance to an EGFR inhibitor by detecting in a sample of tumor cells from a patient for a level of amplification of the epidermal growth factor receptor (EGFR) gene (i.e., the gene encoding EGFR) and/or a level of polysomy of the epidermal growth factor receptor (EGFR) gene or lack thereof as compared to an EGRF inhibitor-sensitive or resistant tumor cell control. The methods of the present invention can include additionally or alternatively detecting in a sample of tumor cells a level of enhanced expression of the E-cadherin protein or transcript, or a level of decreased expression of the ZEB-1 protein or transcript as compared to an EGFR inhibitor-sensitive or resistant tumor cell control.

The present inventors have discovered molecules that predict a response (sensitivity) or resistance to EGFR inhibitors for cancer treatment. In one example, NSCLC cell lines were used as a model to identify potential molecules and to develop strategies that enhance the effect of EGFR inhibitors in NSCLC. Using Western blot analysis and real time RT-PCR, the inventors found expression of E-cadherin in five UCCC cell lines sensitive to EGFR inhibitors. The expression of E-cadherin is inhibited by zinc finger inhibitory proteins. Using real-time RT-PCR, the expression of the zinc-finger transcription factor was found to be elevated in gefitinib-resistant cell lines and its expression was lacking in gefitinib-sensitive ones. Overexpression of E-cadherin in NSCLC cell lines resistant to gefitinib increased their sensitivity. Inducing the expression of E-cadherin either alone or by the HDAC inhibitor, MS-275, in the most resistant cell lines led to an apoptotic effect similar to what is found in cell lines harboring the EGFR mutation. The inventors found that the expression of E-cadherin, and ZEB1 predicts response to EGFR tyrosine kinase inhibitors, and pretreatment with HDAC inhibitors reverses resistance to EGFR inhibitors. In short, the present inventors have evaluated the expression of E-cad and its regulating molecules in NSCLC cell lines, and have found that E-cad expression is lacking or reduced in cell lines resistant to the EGFR inhibitor gefitinib and activated in sensitive cell lines.

The inventors have also discovered that cell lines resistant to EGFR inhibitors have high expression of TF8. In particular, the present inventors have shown the reversal of sensitivity of NSCLC cell lines to gefitinib by restoring E-cad expression and by priming cells with the HDAC inhibitor, MS-275, and by treating cells with combination therapy using EGFR inhibitors and HDAC inhibitors. The present inventors propose herein the first known strategy directed to overcoming resistance to EGFR inhibitors in patients with lung cancer and other types of solid tumors.

The present invention also includes the administration of the combination therapy with EGFR inhibitors and HDAC inhibitors to patients who are predicted to particularly benefit from such treatment, including patients with a history of non-responsiveness to EGFR inhibitors, and patients who are predicted to be less responsive or non-responsive to treatment with EGFR inhibitors (e.g., based on a test to determine resistance or sensitivity). A particularly preferred method for selecting patients who are predicted to be responsive or non-responsive to treatment with EGFR inhibitors is described in PCT Publication No. WO 2005/117553, which is incorporated by reference herein in its entirety. In the present invention, the present inventors propose that these criteria can be used to identify patients that are predicted to benefit from the combination of EGFR inhibitor and HDAC inhibitor. In particular, patients that are predicted to be resistant to (non-responsive to) EGFR inhibitor treatment, as identified using the methods described in PCT Publication No. WO 2005/117553 may particularly benefit from the method of treatment of the present invention. In addition, even patients who are predicted to be likely to respond to (be sensitive to) EGFR inhibitor treatment can also be treated using the method of the present invention.

Specifically, as described in PCT Publication No. WO 2005/117553, the use of combinations of the following markers identify patients that will be sensitive or resistant to EGFR inhibitors: (1) detection of the level of amplification of the epidermal growth factor receptor (EGFR) gene (i.e., the gene encoding EGFR); (2) detection of a level of polysomy of the epidermal growth factor receptor (EGFR) gene; (3) detection of a level of gene amplification of the HER2 gene; (4) detection of the level of polysomy of the HER2 gene; (5) detection of mutations in the EGFR gene; (6) detection of EGFR protein expression; and (7) detection of phosphorylated Akt expression. For example, this publication discloses that patients with tumor cells displaying EGFR gene amplification and/or high polysomy with respect to the EGFR gene (also generally referred to herein as an increase in EGFR gene copy number or a gain in EGFR copy number), and/or HER2 gene amplification and/or high polysomy (also generally referred to herein as an increase in HER2 gene copy number or a gain in HER2 copy number) with respect to the HER2 gene, are predicted to be especially responsive to treatment with EGFR inhibitors, and are therefore the best candidates for the use of this line of therapy. In contrast, patients having tumors with little or no gain in copy number of the EGFR and/or HER2 genes are predicted to have a poor outcome to treatment with EGFR inhibitors. These patients may be particularly good candidates for therapy using the present invention. This publication also discloses that for patients that are EGFR negative (i.e., not predicted to respond to EGFR inhibitors based on EGFR results alone), if such patients' tumors have HER2 gene amplification and/or polysomy (e.g., high trisomy or low or high polysomy) of the HER2 gene, the patient outcome is better as compared to patients without HER2 gene amplification. Furthermore, for patients that are predicted to respond to EGFR inhibitors based on EGFR results alone, HER2 gene amplification and/or high polysomy in these patients' tumors is predictive of even greater sensitivity to the EGFR inhibitor treatment than in the absence of the HER2 gene amplification. This publication also discloses that EGFR protein expression can be used to predict patient outcome with EGFR inhibitor treatment, using assessment criteria that accounts for both expression intensity and the fraction of expression-positive cells in a sample, wherein patients having tumor cells in the upper 50% of the scoring protocol (i.e., denoted positive/high EGFR expressors) had much better outcomes (e.g., better response times, slower progression rates and longer survival times) when treated with EGFR inhibitors than those in the lower expressing groups. Furthermore, PCT Publication No. WO 2005/117553 demonstrated that the combination of detection of EGFR protein expression with HER2 or EGFR gene amplification or polysomy is significantly more predictive of patient outcome to EGFR inhibitor treatment than the detection of one or no markers. Another group of cancer patients with low/no gain of EGFR gene (e.g., “FISH-negative”) and low/no expression of EGFR protein (e.g., “IHC-negative”), which constitute about 30% of the total NSCLC population, seem not to have any clinical benefit (no/very low response rate, short time to progression and short survival time) from EGFR inhibitors. These patients may also be good candidates for treatment using the combination therapy of the present invention. Finally, two other biomarkers, namely mutated EGFR genes or phosphorylated Akt expression, can be combined with any of biomarkers and protocols discussed above to improve the ability to detect patients predicted to respond to EGFR inhibitor treatment. For example, PCT Publication No. WO 2005/117553 demonstrates that the combination of detection of mutations in the EGFR gene with EGFR protein expression, EGFR gene amplification and/or polysomy, and/or HER2 gene amplification and/or polysomy, can be used to select patients who will have clinical benefit from EGFR inhibitor therapy. The combination of the detection of phosphorylated Akt (i.e., activated Akt) with detection of EGFR protein expression and/or detection of EGFR gene amplification and/or polysomy can be used to select patients who will have clinical benefit from EGFR inhibitor therapy. Accordingly, patients selected by any of these criteria to be poor or non-responders to EGFR inhibitor therapy are particularly good candidates for treatment using the method of the invention.

Additionally or alternatively, patients with tumor cells having reduced or absent E-cad expression also show the phenotype of an EGFR inhibitor-resistant cancer and are candidates for the combination therapy as disclosed in the present invention. Additionally or alternatively, patients with tumor cells having activated or enhanced TF-8 expression also show the phenotype of an EGFR inhibitor-resistant cancer and are candidates for the combination therapy as disclosed in the present invention.

However, the present invention is not limited to any of these candidate patients discussed above, since any cancer patient can benefit from the use of the combination therapy disclosed in the present invention.

Various definitions and aspects of the invention will be described below, but the invention is not limited to any specific embodiments that may be used for illustrative or exemplary purposes.

In a first embodiment of the present invention, the present invention includes a method to treat a patient with cancer, comprising administering to the patient a combination of an effective amount of a therapeutic composition comprising at least one histone deacetylase inhibitor and an effective amount of a therapeutic composition comprising at least one EGFR inhibitor. The method also includes a method to treat a patient with a cancer that is resistant to at least one EGFR inhibitor comprising administering to the patient a combination of an effective amount of a therapeutic composition comprising at least one histone deacetylase inhibitor and an effective amount of a therapeutic composition comprising at least one EGFR inhibitor, wherein said cancer is an epithelial malignancy.

The combination may be administered either sequentially or concurrently. Methods of dosing, dosing regimes, and amounts of an EGFR inhibitor and an HDAC inhibitor to administer which are effective to treat cancer are known in the art, and routine optimization may be performed by one skilled in the art to determine preferred dosing methods, regimes, and amounts of each compound to use. Such combination therapy may involve the administration of the HDAC inhibitor before, during, and/or after the administration of the EGFR inhibitor. The administration of the EGFR inhibitor may be separated in time from the administration of HDAC inhibitor by up to several weeks, and may precede it or follow it, but more commonly the administration of the EGFR inhibitor will accompany the administration of the HDAC inhibitor within up to 48 hours, and most commonly within less than 24 hours, including any increment of 30 minutes from 0 to 24 hours and higher (e.g., 30 minutes, 1 hour, 90 minutes, 2 hours, etc.).

In a preferred embodiment, at least a substantial portion of the therapeutic composition comprising at least one histone deacetylase inhibitor is administered before a substantial portion of the therapeutic composition comprising at least one EGFR inhibitor is administered. A substantial portion includes an amount of histone deacetylase inhibitor that is greater than 50% of the total dose to be delivered, and even more preferably includes greater than about 60% of the total dose to be delivered, preferably greater than about 70% of the total dose to be delivered, preferably greater than about 80% of the total dose to be delivered, preferably greater than about 90% of the total dose to be delivered, and most preferably about 100% of the total dose to be delivered. A particularly preferred dosing regime comprises administration of about 100% of the therapeutic composition comprising at least one histone deacetylase inhibitor over a preferred amount of time, followed by administration of about 100% of the therapeutic composition comprising at least one EGFR inhibitor over a preferred amount of time.

Another preferred embodiment includes administering said combination over substantially the same time period, i.e., wherein at least a substantial portion of the therapeutic composition comprising at least one histone deacetylase inhibitor is administered together with a substantial portion of the therapeutic composition comprising at least one EGFR inhibitor. A substantial portion includes an amount of histone deacetylase inhibitor that is greater than 50% of the total dose to be delivered, and even more preferably includes greater than about 60% of the total dose to be delivered, preferably greater than about 70% of the total dose to be delivered, preferably greater than about 80% of the total dose to be delivered, preferably greater than about 90% of the total dose to be delivered, and most preferably about 100% of the total dose to be delivered.

A “therapeutically effective amount” means that amount which, when administered to a mammal, especially a human, for treating a cancer, is sufficient to effect treatment for the cancer. “Treating” or “treatment” of a cancer in a mammal includes one or more of: inhibiting growth of the cancer (e.g., arresting its development), preventing spread of the cancer (e.g., preventing metastases), relieving the cancer (e.g., causing regression of the cancer), preventing recurrence of the cancer, and palliating symptoms of the cancer. As such, a therapeutic benefit or treatment is not necessarily a cure for a particular disease or condition, but rather, preferably encompasses a result which most typically includes alleviation of the disease or condition, elimination of the disease or condition, reduction of a symptom associated with the disease or condition, prevention or alleviation of a secondary disease or condition resulting from the occurrence of a primary disease or condition (e.g., metastatic tumor growth resulting from a primary cancer), and/or prevention of the disease or condition. A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested. According to the present invention, the methods disclosed herein are suitable for use in a patient that is a member of the Vertebrate class, Mammalia, including, without limitation, primates, livestock and domestic pets (e.g., a companion animal). Most typically, a patient will be a human patient.

The EGFR inhibitor and/or the HDAC inhibitor may be administered by any route suitable to the subject being treated and the nature of the subject's condition. Routes of administration include, but are not limited to, administration by injection, including intravenous, intraperitoneal, intramuscular, and subcutaneous injection, by transmucosal or transdermal delivery, through topical applications, nasal spray, suppository and the like or may preferably be administered orally. Formulations may optionally be liposomal formulations, emulsions, formulations designed to administer the drug across mucosal membranes or transdermal formulations. Suitable formulations for each of these methods of administration may be found, for example, in Remington: The Science and Practice of Pharm, 20th ed., A. Gennaro, ed., Lippincott Williams & Wilkins, Philadelphia, Pa., U.S.A. Typical formulations will be either oral or solutions for intravenous infusion. Typical dosage forms will be tablets (for oral administration), solutions for intravenous infusion, and lyophilized powders for reconstitution as solutions for intravenous infusion, although any suitable dosage form is encompassed by the present invention. Kits may contain an HDAC inhibitor and the EGFR inhibitor, also in dosage form, for example packaged together in a common outer packaging.

A therapeutic composition of the present invention may include, in addition to the HDAC inhibitors and/or EGFR inhibitors of the present invention, conventional pharmaceutical excipients, and other conventional, pharmaceutically inactive agents. Additionally, the compositions may include active agents in addition to the HDAC inhibitors and/or EGFR inhibitors of the present invention. These additional active agents may include one or more other pharmaceutically active agents. The compositions may be in gaseous, liquid, semi-liquid or solid form, formulated in a manner suitable for the route of administration to be used. For oral administration, capsules and tablets are typically used. For parenteral administration, reconstitution of a lyophilized powder, prepared as described herein, is typically used. The compositions may further comprise: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to form a solution or suspension. If desired, the pharmaceutical composition to be administered may also contain minor amounts of auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known in the art, or will be apparent, to those skilled in this art. The composition or formulation to be administered will, in any event, contain a sufficient quantity of a HDAC inhibitor and/or EGFR inhibitor of the present invention to reduce such activity in vivo, thereby treating the disease state of the subject.

Dosage forms or compositions may optionally comprise one or more of an HDAC inhibitor and/or EGFR inhibitor according to the present invention in the range of 0.005% to 100% (weight/weight) with the balance comprising additional substances such as those described herein. For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium croscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art. The compositions may optionally contain 0.01%-100% (weight/weight) of one or more of an HDAC inhibitor and/or EGFR inhibitor of the present invention; optionally 0.1-95%, and optionally 1-95%.

Salts, preferably sodium salts, of an HDAC inhibitor and/or EGFR inhibitor of the present invention may be prepared with carriers that protect the compound against rapid elimination from the body, such as time release formulations or coatings. The formulations may further include other active compounds to obtain desired combinations of properties. Oral pharmaceutical dosage forms may be as a solid, gel or liquid. Examples of solid dosage forms include, but are not limited to tablets, capsules, granules, and bulk powders. More specific examples of oral tablets include compressed, chewable lozenges and tablets that may be enteric-coated, sugar-coated or film-coated. Examples of capsules include hard or soft gelatin capsules. Granules and powders may be provided in non-effervescent or effervescent forms. Each may be combined with other ingredients known to those skilled in the art. In certain embodiments, HDAC inhibitors according to the present invention are provided as solid dosage forms, preferably capsules or tablets. The tablets, pills, capsules, troches and the like may optionally contain one or more of the following ingredients, or compounds of a similar nature: a binder; a diluent; a disintegrating agent; a lubricant; a glidant; a sweetening agent; and a flavoring agent. Examples of binders that may be used include, but are not limited to, microcrystalline cellulose, gum tragacanth, glucose solution, acacia mucilage, gelatin solution, sucrose and starch paste. Examples of lubricants that may be used include, but are not limited to, talc, starch, magnesium or calcium stearate, lycopodium and stearic acid. Examples of diluents that may be used include, but are not limited to, lactose, sucrose, starch, kaolin, salt, mannitol and dicalcium phosphate. Examples of glidants that may be used include, but are not limited to, colloidal silicon dioxide. Examples of disintegrating agents that may be used include, but are not limited to, croscarmellose sodium, sodium starch glycolate, alginic acid, corn starch, potato starch, bentonite, methylcellulose, agar and carboxymethylcellulose. Examples of coloring agents that may be used include, but are not limited to, any of the approved certified water soluble FD and C dyes, mixtures thereof, and water insoluble FD and C dyes suspended on alumina hydrate. Examples of sweetening agents that may be used include, but are not limited to, sucrose, lactose, mannitol and artificial sweetening agents such as sodium cyclamate and saccharin, and any number of spray-dried flavors. Examples of flavoring agents that may be used include, but are not limited to, natural flavors extracted from plants such as fruits and synthetic blends of compounds that produce a pleasant sensation, such as, but not limited to peppermint and methyl salicylate. Examples of wetting agents that may be used include, but are not limited to, propylene glycol monostearate, sorbitan monooleate, diethylene glycol monolaurate and polyoxyethylene lauryl ether. Examples of anti-emetic coatings that may be used include, but are not limited to, fatty acids, fats, waxes, shellac, ammoniated shellac and cellulose acetate phthalates. Examples of film coatings that may be used include, but are not limited to, hydroxyethylcellulose, sodium carboxymethylcellulose, polyethylene glycol 4000 and cellulose acetate phthalate. If oral administration is desired, the salt of the compound may optionally be provided in a composition that protects it from the acidic environment of the stomach. For example, the composition can be formulated in an enteric-coating that maintains its integrity in the stomach and releases the active compound in the intestine. The composition may also be formulated in combination with an antacid or other such ingredient. Compounds according to the present invention may also be administered as a component of an elixir, suspension, syrup, wafer, sprinkle, chewing gum or the like. A syrup may optionally comprise, in addition to the active compounds, sucrose as a sweetening agent and certain preservatives, dyes and colorings and flavors.

The HDACs are a family including at least eighteen enzymes, grouped in three classes (Class I, II and III). Class I HDACs include, but are not limited to, HADCs 1, 2, 3, and 8. Class I HDACs can be found in the nucleus and are believed to be involved with transcriptional control repressors. Class II HDACs include, but are not limited to, HDACS 4, 5, 6, 7, and 9 and can be found in both the cytoplasm as well as the nucleus. Class III HDACs are believed to be NAD dependent proteins and include, but are not limited to, members of the Sirtuin family of proteins. Non-limiting examples of sirtuin proteins include SIRT1-7. As used herein, the term “selective HDAC” refers to an HDAC inhibitor that does not interact with all three HDAC classes.

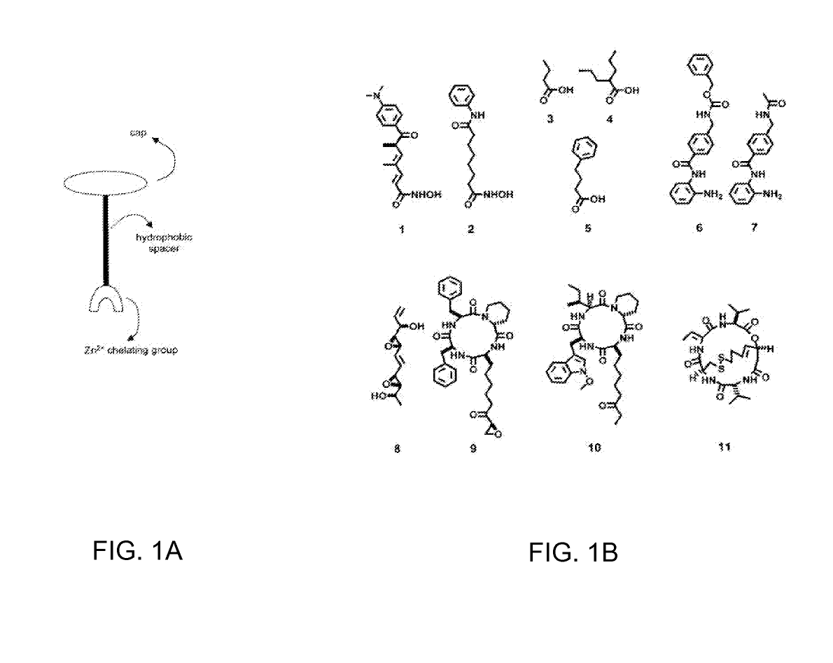

HDAC inhibitors can be classified broadly into pan HDAC inhibitors and selective HDAC inhibitors. Although there is a large structural diversity of known HDAC inhibitors, they share common features: a part that interacts with the enzyme active site and a side-chain that sits inside the channel leading to the active site. This can be seen with the hydroxamates such as SAHA, where the hydroxamate group is believed to interact with the active site. In the case of the depsipeptides, it is believed that an intracellular reduction of the disulphide bond creates a free thiol group (which interacts with the active site) attached to a 4-carbon alkenyl chain. A difference between the HDAC inhibitors is in the way that they interact with the rim of the HDAC channel, which is at the opposite end of the channel to the active site. It is this interaction, between the HDAC inhibitor and the rim of the channel, which is believed to account, at least in part, for some observed differences in HDAC selectivity between pan-HDAC inhibitors, such as SAHA and selective HDAC inhibitors such as the depsipeptides. An HDAC inhibitor-containing therapeutic composition compatible with the methods of the present invention includes a composition comprising an HDAC inhibitor such as, for example, hydroxamic acids such as suberoylanilidine hydroxamic acid, TSA, TSC, m-carboxycinnamic acid bishydroxamide (CBHA), pyrozamide, salicylbishyudoxamic acid, suberoyl bishydroxamic acid (SBHA), azelaic bishydroxamic acid (ABHA), Azelaic-1-hydroxamate-9-anilide (AAHA), Oxamflatin, Scriptaid, CHAP, MW2996, MW2976, any of the hydroxamic acids disclosed in U.S. Pat. Nos. 5,369,108, 5,932,616, 5,700.811, 6,087,367, 6,511,990 (each of which are incorporated by reference in their entirety), and SAHA (NVP-LAQ-824, PXD-1-1); carboxylic acids such as butanoic, valproic, and 4-phenylbutanoic acids; benzamides and derivatives such as N-acetyldinaline, CI-994 and MS-275; epoxides and cyclic tetrapeptides such as trapoxins, depeudecin, depsipeptide FK 228, FR225497, Apicidin cyclic tetrapeptide, Apicidin Ia, Apicidin Ib, Apicidin Ic, Apicidin Ia, Apicidin IIb; short-chain fatty acids and derivatives such as sodium butyrate, isovalerate, valerate. 4-phenylbutyrate (4-PBA), phenylbutyrate (PB) propionate, butyramide, isobutyramide, phylacetate, 3-bromopropionate, tributyrin, valproic acid, Valproate, and Pivanex™; a cyclic tetrapeptide containing a 2-amino-8-oxo-9,10-epoxy-decanoyl moiety, and a cyclic peptide without the 2-amino-8-oxo-9,10-epoxy-decanoyl moiety. See FIG. 1A and FIG. 1B. A particularly preferred HDAC inhibitor is MS-275. Further HDAC inhibitors include Savicol®, Baceca®, MGCD0103, MG98 (an antisense molecule), and LBH589.

Preferred amounts of HDAC inhibitor to administer may be chosen by one of skill in the art, and include amounts known in the art to be efficacious for treating cancers. Examples of suitable methods to treat cancer with HDAC inhibitors and suitable amounts of HDAC inhibitors to use are known in the art, such as, for example, in U.S. Patent Publication 20040132825, U.S. Ser. No. 10/692,523, Bacopoulos et al., entitled METHODS OF TREATING CANCER WITH HDAC INHIBITORS, filed Oct. 24, 2003, which is incorporated herein by reference in its entirety. Suitable dosing for an HDAC inhibitor includes dosing already established for that HDAC inhibitor, as described in such documents as those listed herein and as known in the art. A preferred amount to administer for MS-275, for example, includes a minimum of about 0.01 milligram per meter squared (mg/m²) and a maximum of about 1,000 mg/m², and can include ranges between: about 0.1 mg and about 100 mg, about 0.2 mg and about 90 mg, about 0.3 mg/m² and about 70 mg/m², about 0.4 mg/m² and about 50 mg/m², about 0.5 mg/m² and about 30 mg/m², about 0.6 mg/m² and about 20 mg/m², about 0.7 mg/m² and about 15 mg/m², about 0.8 mg/m² and about 10 mg/m², about 0.9 mg/m² and about 5 mg/m². Other preferred amounts to administer include about 0.1 mg/m², about 0.5 mg/m², about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², and about 7.5 mg/m². The dosing can occur over any time period, for example daily, every 2-6 days, biweekly, monthly, or in one aspect, weekly. In preferred embodiments, one may administer HDAC inhibitory compounds of the present invention orally, although one can also administer by intravenous and intramuscular injection. In one embodiment, an HDAC inhibitor such as MS-275 is administered at 2 mg/m² orally weekly for 3 out of 4 weeks or 4 mg/m² orally biweekly.

Provided herein is a method to treat a patient with cancer, comprising administering to the patient at least one HDAC inhibitor and at least one kinase inhibitor with anti-EGFR activity. In one embodiment, the HDAC inhibitor is a selective HDAC inhibitor. One non-limiting example of a selective HDAC inhibitor is MS-275. Other selective HDAC inhibitors are known and can be used as described herein. In some embodiments, the kinase inhibitor with anti-EGFR activity is a dual kinase inhibitor (e.g., has dual kinase specificity).

An EGFR inhibitor-containing therapeutic composition compatible with the methods of the present invention includes a composition comprising an EGFR inhibitor. Tyrosine kinase inhibitors are also contemplated herein. Currently there are two main classes of EGFR inhibitors: anti-EGFR family tyrosine kinase inhibitors (small molecules) and anti-EGFR monoclonal antibodies. Both categories are contemplated within the meaning of EGFR inhibitor used herein. Examples of small molecules include EGFR-specific and reversible inhibitors such as, for example, gefitinib (IRESSA®, ZD1839), erlotinib (TARCEVA®, OSI-774, CP-358), or PKI-166; EGFR-specific and irreversible inhibitors, such as EKI-569; a PAN-HER (human EGF receptor family) reversible inhibitor, such as GW2016 (targets both EGFR and Her2/neu); and a PAN-HER irreversible inhibitor, such as CI-1033 (4-anilinoquinazoline).

Further examples of tyrosine kinase inhibitors and EGFR antagonists include, but are not limited to, small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037 (each of which are incorporated by reference herein in their entireties). Additional small molecule EGFR antagonists include, but are not limited to, PD 183805 (CI 1033, 2-propenamide, N-[4-[(3-chloro-4-fluorophenyl)amino]-7-[3-(4-morpholinyl)propoxy]-6-quin-azolinyl]-, dihydrochloride, Pfizer Inc.); ZM 105180 ((6-amino-4-(3-methylphenyl-amino)-quinazoline, Zeneca); BIBX-1382 (N-8-(3-chloro-4-fluoro-phenyl)-N-2-(1-methyl-piperidin-4-yl)-pyrimido[5,-4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol)-; (R)-6-(4-hydroxyphenyl)-4-[(1-phenylethyl)amino]-7H-pyrrolo[2,3-d]pyrimi-dine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(-dimethylamino)-2-butenamide) (Wyeth); Imatinib; STI-571; LFM-A13; PD153035; Piceatannol; PP1, Lapatinib (Tykerb®, GW572016, GlaxoSmithKline); AEE788; SU4132; SU6656; Semazanib; SU6668, ZD6126 AG1478 (Sugen); Vandetanib (ZACTIMA™; ZD6474) and AG1571 (SU 5271; Sugen). Further examples of EGFR and HER family antagonists or inhibitors will be known in the art and are also contemplated herein.

Examples of monoclonal antibodies and antibody variants, fusions, derivatives, and fragments thereof include C225 (CETUXIMAB; ERBITUX.RTM.), ABX-EGF (human) (Abgenics, San Francisco, Calif.), EMD-72000 (humanized), h-R3 (humanized), and MDX-447 (bi-specific, EGFR-CK64); MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; and mAb 806 or humanized mAb 806 (Johns et al., J. Biol. Chem. 279(29):30375-30384 (2004)).

The anti-EGFR antibody may be conjugated with a cytotoxic agent, thus generating an immunoconjugate (see, e.g., EP659,439A2, Merck Patent GmbH). Additionally, fusion proteins, single chain antibodies, and fragments or variants thereof based upon the antibodies and epitope binding regions of the antibodies described above are also contemplated herein. The construction of such polypeptides, fusion proteins, and single chain antibodies is known in the art and can include, but is not limited to, conventional recombinant techniques

Therapeutic compositions also include a drug having substantially the same biological activity as gefitinib, lapatinib and erlotinib. A particularly preferred EGFR inhibitor is gefitinib and/or erlotinib and/or lapatanib. Preferred amounts of EGFR inhibitor to administer may be chosen by one of skill in the art, and include amounts known in the art to be efficacious for treating other cancers. Suitable dosing for an EGFR inhibitor will be the dosing already established for that EGFR inhibitor, as described in such documents as those listed below and known in the art. Examples of suitable methods to treat cancer with EGFR inhibitors and suitable amounts of EGFR inhibitors to use are known in the art, such as, for example, in U.S. Patent Publication 20030114504, U.S. Ser. No. 10/228,544 (each of which are incorporated by reference in their entireties), Webster et al., entitled COMPOSITIONS AND METHODS FOR TREATMENT OF CANCER, filed Aug. 27, 2002, which is incorporated herein by reference in its entirety. A preferred amount to administer or treat with includes a minimum of about 5 mg and a maximum of about 20,000 mg, and can include ranges between: about 20 mg and about 15,000 mg, about 40 mg and about 10,000 mg, about 80 mg and about 5000 mg, about 120 mg and about 2000 mg, about 180 mg and about 1500 mg, about 200 mg and about 1000 mg, about 250 mg and about 800 mg, about 300 mg and about 700 mg, about 400 mg and about 600 mg. Other preferred amounts include about 10 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1200 mg, about 1400 mg, about 1600 mg, about 1800 mg, about 2000 mg, about 2200 mg, about 2400 mg, about 2600 mg, about 2800 mg, about 3000 mg, about 3500 mg, about 4000 mg, about 4500 mg, about 5000 mg, about 5500 mg, about 6000 mg, about 6500 mg, about 7000 mg, about 8000 mg, about 10,000 mg, about 12,000 mg, and about 15,000 mg. The dosing will be over any time period, preferably monthly, more preferably weekly, and even more preferably daily.

Provided herein is a method to treat a patient with cancer, comprising administering to the patient at least one HDAC inhibitor and at least one EGFR inhibitor. In one embodiment, the HDAC inhibitor is a selective HDAC inhibitor. One non-limiting example of a selective HDAC inhibitor is MS-275. Other selective HDAC inhibitors are known and described herein. In another embodiment, the EGFR inhibitor is a small molecule EGFR inhibitor. Non-limiting examples of small-molecule EGFR inhibitors include gefitinib, erlotinib, and lapatinib. In another embodiment, the EGFR inhibitor is an antibody EGFR inhibitor. In a further embodiment, the HDAC inhibitor is a selective HDAC inhibitor and the EGFR inhibitor is erlotinib, gefitinib, or lapatinib.

In one embodiment, one may administer EGFR inhibitory compounds of the present invention orally, although one can also administer them by intravenous and intramuscular injection. In one embodiment, an EGFR inhibitor is gefitinib and is administered orally in a bolus of about 2,000 mg once per week. In another embodiment, the EGFR inhibitor is gefitinib and is administered daily at about 250 mg per day. In another embodiment, the inhibitor is erlotinib and is administered orally at about 150 mg per day.

Periods of time in which to administer any HDAC inhibitors and/or EGFR inhibitors are either known in the art and/or may be determined by one of skill in the art, and include for about a day, for about 2 days, for about 3 days, for about 4 days, for about 5 days, for about 6 days, for about a week, for about a week and a half, for about 2 weeks, for about 2 and a half weeks, for about 3 weeks, for about three and a half weeks, for about 4 weeks, for about 5 weeks, for about 6 weeks, for about 8 weeks, for about 10 weeks, for about 15 weeks, for about 20 weeks, for about 25 weeks, for about 30 weeks, for about 40 weeks, and for about 52 weeks. The HDAC inhibitors and/or EGFR inhibitors may be optionally administered over successive periods of time with one or more rest periods (i.e., no administration of HDAC inhibitors and/or EGFR inhibitors). Rest periods again are either known in the art and/or may be determined by one of skill in the art, and include for about a day, for about 2 days, for about 3 days, for about 4 days, for about 5 days, for about 6 days, for about a week, for about a week and a half, for about 2 weeks, for about 2 and a half weeks, for about 3 weeks, for about three and a half weeks, for about 4 weeks, for about 5 weeks, for about 6 weeks, for about 8 weeks, for about 10 weeks, for about 15 weeks, for about 20 weeks, for about 25 weeks, for about 30 weeks, for about 40 weeks, and for about 52 weeks.

Preferred cancers to treat with the methods of the present invention include cancers that are epithelial malignancies (having epithelial origin), and particularly any cancers (tumors) that express EGFR. A preferred cancer to treat is a cancer that is resistant to EGFR inhibitors and in one aspect, can be an epithelial malignancy that is resistant to EGFR inhibitors. In an EGFR inhibitor-resistant cancer, the cancer can include tumors (cancerous cells) with little or no gain in copy number (low/no gene amplification or polysomy), tumors that are low expressors of EGFR protein (in the lower 50% of an appropriate scoring protocol, as in PCT Publication No. WO 2005/117553), or especially a combination of low/no gain of EGFR gene and low/no expression of EGFR protein. EGFR-resistant cancers can also include tumors that have low/no gain in EGFR and are P-Akt positive, or tumors with EGFR gene amplification and/or polysomy, but that are P-Akt negative. EGFR-resistant cancers can also include tumors without mutations in EGFR that meet one or more of the other criteria for poor or non-responders as discussed above. Non-limiting examples of premalignant or precancerous cancers/tumors having epithelial origin include actinic keratoses, arsenic keratoses, xeroderma pigmentosum, Bowen's disease, leukoplakias, metaplasias, dysplasias and papillomas of mucous membranes, e.g. of the mouth, tongue, pharynx, esophagus, gastro-esophageal junction and larynx, precancerous changes of the bronchial mucous membrane such as metaplasias and dysplasias (especially frequent in heavy smokers and people who work with asbestos and/or uranium), dysplasias and leukoplakias of the cervix uteri, vulval dystrophy, esophagus, gastro-esophageal junction precancerous changes of the bladder, e.g. metaplasias and dysplasias, papillomas of the bladder as well as polyps of the intestinal tract. Non-limiting examples of semi-malignant or malignant cancers/tumors of the epithelial origin are breast cancer, skin cancer (e.g., basal cell carcinomas), bladder cancer (e.g., superficial bladder carcinomas), colon cancer, gastro-intestinal (GI) cancer, prostate cancer, uterine cancer, cervical cancer, ovarian cancer, esophageal cancer, stomach cancer, laryngeal cancer and lung cancer.

Provided herein is a method of treating cancer having an epithelial origin comprising administering to the patient a combination of at least one HDAC inhibitor and lapatinib. Non-limiting examples of cancers having epithelial origin include breast cancer, skin cancer, bladder cancer, colon cancer, prostate cancer, uterine cancer, cervical cancer, ovarian cancer, esophageal cancer, stomach cancer, gastrointestinal cancer (GI), pancreatic cancer, laryngeal cancer, and lung cancer.

In another preferred EGFR-resistant cancer, the cancer preferably comprises cancerous cells having a reduced level of E-cadherin gene expression compared to cancerous cells that are sensitive to EGFR inhibitors. In yet another preferred EGFR-resistant cancer, the cancer preferably comprises cancerous cells having an enhanced level of zinc finger transcription factors expression compared to cancerous cells that are sensitive to EGFR inhibitors. A preferred zinc finger transcription factor is TF8. Another preferred type of cancer to treat is a lung cancer, and particularly preferred is a lung cancer that is derived from an epithelial cell, such as non-small cell lung cancer.

The methods of the present invention also include a method to treat a patient with an EGFR inhibitor-resistant cancer comprising the step of sensitizing the cancer cells resistant to at least one EGFR inhibitor comprising administering to the patient a combination of an effective amount of a therapeutic composition comprising at least one histone deacetylase (HDAC) inhibitor and an effective amount of a therapeutic composition comprising at least one EGFR inhibitor.

The methods of the present invention can also include an additional step comprising the step of evaluating the cancer to predict sensitivity to or for resistance to EGFR inhibitors. The method can include evaluating any of the markers described above that are predictive of poor or non-responsiveness to EGFR inhibitor therapy. For example, in one embodiment, the step of evaluating the cancer for sensitivity or resistance to an EGFR inhibitor comprises: a) detecting in a sample of tumor cells from a patient to be tested a level of amplification of the epidermal growth factor receptor (EGFR) gene and/or a level of polysomy of the epidermal growth factor receptor (EGFR) gene; b) comparing the level of EGFR gene amplification and/or polysomy in the tumor cell sample to a control level of EGFR gene amplification and/or polysomy selected from the group consisting of: i) a control level that has been correlated with sensitivity to an EGFR inhibitor; and ii) a control level that has been correlated with resistance to an EGFR inhibitor; and c) selecting the patient as being predicted to benefit from therapeutic administration of the combination, if the level of EGFR gene amplification and/or polysomy in the patient's tumor cells is decreased relative to the control level of EGFR gene amplification and/or polysomy that has been correlated with sensitivity to EGFR inhibitor, or if the level of EGFR gene amplification and/or polysomy in the patient's tumor cells is statistically similar than the level of EGFR gene amplification and/or polysomy that has been correlated with resistance to an EGFR inhibitor. Other similar steps of evaluating the tumor can be performed based on the criteria discussed herein.

In another embodiment, the step of evaluating the cancer for sensitivity or resistance to an EGFR inhibitor may additionally or alternately comprise detecting in the sample of tumor cells a level of expression of the E-cadherin protein; comparing the level of E-cadherin expression in the tumor cell sample to a control level of E-cadherin expression being either a control level that has been correlated with sensitivity to an EGFR inhibitor or a control level that has been correlated with resistance to an EGFR inhibitor; and selecting the patient as being predicted to benefit from therapeutic administration of combination, if the level of E-cadherin expression in the patient's tumor cells is statistically reduced compared to the control level of E-cadherin expression that has been correlated with sensitivity to an EGFR inhibitor, or if the level of E-cadherin expression in the patient's tumor cells is statistically similar than the level of E-cadherin expression that has been correlated with resistance to an EGFR inhibitor.

In another embodiment, the step of evaluating the cancer for sensitivity or resistance to an EGFR inhibitor may additionally or alternately comprise detecting in the sample of tumor cells a level of expression of at least one component of TF8; comparing the level at least one component of TF8's expression in the tumor cell sample to a control level of at least one component of TF8's expression being either: a control level that has been correlated with sensitivity to an EGFR inhibitor, or a control level that has been correlated with resistance to an EGFR inhibitor; and selecting the patient as being predicted to benefit from therapeutic administration of combination, if the level of at least one component of TF8's expression in the patient's tumor cells is statistically increased compared to the control level of at least one component of TF8's expression that has been correlated with sensitivity to an EGFR inhibitor, or if the level of at least one component of TF8's expression in the patient's tumor cells is statistically similar than the level of at least one component of TF8's expression that has been correlated with resistance to an EGFR inhibitor. A preferred component of TF8 to detect is ZEB.

Suitable methods of obtaining a patient sample are known to a person of skill in the art. A patient sample can include any bodily fluid or tissue from a patient that may contain tumor cells or proteins of tumor cells. More specifically, according to the present invention, the term “test sample” or “patient sample” can be used generally to refer to a sample of any type which contains cells or products that have been secreted from cells to be evaluated by the present method, including but not limited to, a sample of isolated cells, a tissue sample and/or a bodily fluid sample. Most typically in the present invention, the sample is a tissue sample. According to the present invention, a sample of isolated cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. The cells in the cell sample are not necessarily of the same type, although purification methods can be used to enrich for the type of cells that are preferably evaluated. Cells can be obtained, for example, by scraping of a tissue, processing of a tissue sample to release individual cells, or isolation from a bodily fluid.

A tissue sample, although similar to a sample of isolated cells, is defined herein as a section of an organ or tissue of the body which typically includes several cell types and/or cytoskeletal structure which holds the cells together. One of skill in the art will appreciate that the term “tissue sample” may be used, in some instances, interchangeably with a “cell sample”, although it is preferably used to designate a more complex structure than a cell sample. A tissue sample can be obtained by a biopsy, for example, including by cutting, slicing, or a punch.

A bodily fluid sample, like the tissue sample, contains the cells to be evaluated, and is a fluid obtained by any method suitable for the particular bodily fluid to be sampled. Bodily fluids suitable for sampling include, but are not limited to, blood, mucous, seminal fluid, saliva, breast milk, bile and urine.

In general, the sample type (i.e., cell, tissue or bodily fluid) is selected based on the accessibility and structure of the organ or tissue to be evaluated for tumor cell growth and/or on what type of cancer is to be evaluated. For example, if the organ/tissue to be evaluated is the breast, the sample can be a sample of epithelial cells from a biopsy (i.e., a cell sample) or a breast tissue sample from a biopsy (a tissue sample). The present invention is particularly useful for evaluating patients with lung cancer and particularly, non-small cell lung carcinoma, and in this case, a typical sample is a section of a lung tumor from the patient.

The copy number of genes in tumor cells according to the invention can be measured in primary tumors, metastatic tumors, locally recurring tumors, ductal carcinomas in situ, or other tumors. The markers can be measured in solid tumors that are fresh, frozen, fixed or otherwise preserved. They can be measured in cytoplasmic or nuclear tumor extracts; or in tumor membranes including but not limited to plasma, mitochondrial, golgi or nuclear membranes; in the nuclear matrix; or in tumor cell organelles and their extracts including but not limited to ribosomes, nuclei, mitochondria, golgi.

Once a sample is obtained from the patient, the sample is evaluated for sensitivity or resistance to EGFR inhibitors as disclosed herein. In some embodiments of the present invention, a tissue, a cell or a portion thereof (e.g., a section of tissue, a component of a cell such as nucleic acids, etc.) is contacted with one or more nucleic acids. Such methods can include cell-based assays or non-cell-based assays. The tissue or cell expressing a target gene is typically contacted with a detection agent (e.g., a probe, primer, or other detectable marker), by any suitable method, such as by mixing, hybridizing, or combining in a manner that allows detection of the target gene by a suitable technique.

The patient sample is prepared by any suitable method for the detection technique utilized. In one embodiment, the patient sample can be used fresh, frozen, fixed or otherwise preserved. For example, the patient tumor cells can be prepared by immobilizing patient tissue in, for example, paraffin. The immobilized tissue can be sectioned and then contacted with a probe for detection of hybridization of the probe to a target gene.

In a preferred embodiment, detection of a gene according to the present invention is accomplished using hybridization assays. Nucleic acid hybridization simply involves contacting a probe (e.g., an oligonucleotide or larger polynucleotide) and target nucleic acid under conditions where the probe and its complementary target can form stable hybrid duplexes through complementary base pairing. As used herein, hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety. Nucleic acids that do not form hybrid duplexes are washed away from the hybridized nucleic acids and the hybridized nucleic acids can then be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

High stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). One of skill in the art can use the formulae in Meinkoth et al., 1984, Anal. Biochem. 138, 267-284 (incorporated herein by reference in its entirety) to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C., more preferably, between about 28° C. and about 40° C., and even more preferably, between about 35° C. and about 45° C. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62.

The hybridized nucleic acids are detected by detecting one or more labels attached to the sample nucleic acids. The labels may be incorporated by any of a number of means well known to those of skill in the art. Detectable labels suitable for use in the present invention include any composition detectable by spectroscopic, photochemical, biochemical, immunochemical, electrical, optical or chemical means. Useful labels in the present invention include fluorescent dyes (e.g., fluorescein, texas red, rhodamine, green fluorescent protein, and the like), radiolabels (e.g., ³H, ¹²⁵I, ³⁵S, ¹⁴C, or ³²P), and colorimetric labels. Means of detecting such labels are well known to those of skill in the art. Thus, for example, radiolabels may be detected using photographic film or scintillation counters, fluorescent markers may be detected using a photodetector to detect emitted light. Colorimetric labels are detected by simply visualizing the colored label. Preferably, the hybridizing nucleic acids are detected by fluorescent labels and most preferably, in the context of a FISH assay.

In accordance with the present invention, an isolated polynucleotide, or an isolated nucleic acid molecule, is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. Polynucleotides such as those used in a method of the present invention to detect genes (e.g., by hybridization to a gene) are typically a portion of the target gene that is suitable for use as a hybridization probe or PCR primer for the identification of a full-length gene (or portion thereof) in a given sample (e.g., a cell sample). An isolated nucleic acid molecule can include a gene or a portion of a gene (e.g., the regulatory region or promoter). An isolated nucleic acid molecule that includes a gene is not a fragment of a chromosome that includes such gene, but rather includes the coding region and regulatory regions associated with the gene, but no additional genes naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. Preferably, an isolated nucleic acid molecule of the present invention is produced using recombinant DNA technology (e.g., polymerase chain reaction (PCR) amplification, cloning) or chemical synthesis. If the polynucleotide is an oligonucleotide probe, the probe typically ranges from about 5 to about 50 or about 500 nucleotides, or from about 10 to about 40 nucleotides, or from about 15 to about 40 nucleotides in length, or any range of length in between 10 and 1000 nucleotides, in whole integer increments (i.e., 10, 11, 12, 13 . . . 999, 1000).

According to the present invention, a probe is a nucleic acid molecule which typically ranges in size from about 8 nucleotides to several hundred nucleotides in length as discussed above. Such a molecule is typically used to identify a target nucleic acid sequence in a sample by hybridizing to such target nucleic acid sequence under stringent hybridization conditions. Hybridization conditions have been described in detail above.

PCR primers are also nucleic acid sequences, although PCR primers are typically oligonucleotides of fairly short length which are used in polymerase chain reactions. PCR primers and hybridization probes can readily be developed and produced by those of skill in the art, using sequence information from the target sequence. (See, for example, Sambrook et al, supra or Glick et al., supra).

In one embodiment, the method of the invention can also include a step of detecting whether there is a change (regulation, modification) in the level of expression of E-cad and/or a component of TF8, such as, for example ZEB1 in the cell. As used herein, the term “expression,” can refer to detecting transcription of the gene and/or to detecting translation of the protein encoded by the gene. To detect expression of a gene or protein refers to the act of actively determining whether a gene or protein is expressed or not. This can include determining whether the expression is upregulated as compared to a control, downregulated as compared to a control, or unchanged as compared to a control. Expression of transcripts and/or proteins is measured by any of a variety of known methods in the art. For RNA expression, methods include but are not limited to: extraction of cellular mRNA and Northern blotting using labeled probes that hybridize to transcripts encoding all or part of one or more of the genes of this invention; amplification of mRNA expressed from one or more of the genes of this invention using gene-specific primers, polymerase chain reaction (PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene. Measurement of translation of a protein include any suitable method for detecting and/or measuring proteins from a cell or cell extract. Such methods include, but are not limited to, immunoblot (e.g., Western blot), enzyme-linked immunosorbant assay (ELISA), radioimmunoassay (RIA), immunoprecipitation, immunohistochemistry (IHC), immunofluorescence, fluorescence activated cell sorting (FACS) and immunofluorescence microscopy.

The nucleotide sequence of the human epidermal growth factor receptor (EGFR); E-cadherin; and TF8 genes are known in the art and can be found under GenBank Accession No. AY588246 (incorporated herein by reference), for example. Nucleotide probes and antibodies are also known in the art and available for use as probes to detect EGFR, E-cadherin, and TF8 (ZEB1) genes and proteins.

In the method of the invention, the level of EGFR gene amplification and/or polysomy in the tumor cell sample is compared to a control level of EGFR gene amplification and/or polysomy selected from: (i) a control level that has been correlated with sensitivity to EGFR inhibitor; and (ii) a control level that has been correlated with resistance to EGFR inhibitor. A patient is selected as being predicted to benefit from therapeutic administration of a combination therapy of the present invention, if the level of EGFR gene amplification and/or polysomy in the patient's tumor cells is statistically similar to the control level of EGFR gene amplification and/or polysomy that has been correlated with resistance to EGFR inhibitor, or if the level of EGFR gene amplification and/or polysomy in the patient's tumor cells is statistically less than or reduced from the level of EGFR gene amplification and/or polysomy that has been correlated with sensitivity to EGFR inhibitor.

In another alternate or additional method of the invention, the level of E-cadherin expression in the tumor cell sample may be compared to a control level of E-cadherin expression selected from: (i) a control level that has been correlated with sensitivity to EGFR inhibitor; and (ii) a control level that has been correlated with resistance to EGFR inhibitor. A patient is selected as being predicted to benefit from therapeutic administration of a combination therapy of the present invention, if the level of E-cadherin expression in the patient's tumor cells is statistically similar to the control level of E-cadherin expression that has been correlated with resistance to EGFR inhibitor, or if the level of E-cadherin expression in the patient's tumor cells is statistically less than or reduced from the level of E-cadherin expression that has been correlated with sensitivity to EGFR inhibitor.

In another alternate or additional method of the invention, the level of a component of TF8, preferably ZEB1, expression in the tumor cell sample may be compared to a control level of a TF8 component's expression selected from: (i) a control level that has been correlated with sensitivity to EGFR inhibitor; and (ii) a control level that has been correlated with resistance to EGFR inhibitor. A patient is selected as being predicted to benefit from therapeutic administration of a combination therapy of the present invention, if the level of a TF8 component's expression in the patient's tumor cells is statistically similar to the control level of a TF8 component's expression that has been correlated with resistance to EGFR inhibitor, or if the level of a TF8 component's expression in the patient's tumor cells is statistically greater than or enhanced from the level of a TF8 component's expression that has been correlated with sensitivity to EGFR inhibitor.

More specifically, according to the present invention, a “control level” is a control level of gene amplification and/or polysomy, and/or gene transcription or translation, which can include a level that is correlated with sensitivity to EGFR inhibitor or a level that is correlated with resistance to EGFR inhibitor. Therefore, it can be determined, based on the control or baseline level of gene amplification and/or polysomy, whether a patient sample is more likely to be sensitive to or resistant to EGFR inhibitor therapy. In one embodiment, patients are classified into six categories with ascending number of copies per cell: (1) Disomy (≦2 copies of both targets in >90% of cells); (2) Low trisomy (≦2 copies of the gene in ≧40% of cells and 3 copies in 10-40% of the cells); (3) High trisomy (≦2 copies of the gene in ≧40% of cells and 3 copies in ≧40% of cells); (4) Low polysomy (≧4 copies of the gene in 10-40% of cells); (5) High polysomy (≧4 copies of the gene in ≧40% of cells); and (6) Gene Amplification (GA), defined by presence of tight EGFR gene clusters and a ratio gene/chromosome per cell ≧2, or an average of ≧15 copies of EGFR per cell in ≧10% of analyzed cells. The present inventors have found that patients with high gene copy numbers or a gain in copy numbers (e.g., gene amplification and/or polysomy including high trisomy, low polysomy or high polysomy) of EGFR and/or HER2 are more likely to have a higher response rate to EGFR inhibitor therapy, a lower rate of progressive disease, a longer time to progression, and a higher rate of long term survivors. The higher the polysomy or overall gain in gene copy number, the better the predicted outcome. The present inventors found that the presence of HER2 gene amplification and/or polysomy in patient tumor cells confers a more sensitive phenotype to EGFR positive patients (e.g., patients showing a gain in EGFR gene copy numbers) and a better outcome to EGFR negative patients (e.g., patients having no or low gain in EGFR gene copy numbers).

The method for establishing a control level of gene amplification, polysomy and/or gene transcription or translation, is selected based on the sample type, the tissue or organ from which the sample is obtained, and the status of the patient to be evaluated. Preferably, the method is the same method that will be used to evaluate the sample in the patient. In a preferred embodiment, the control level is established using the same cell type as the cell to be evaluated. In a preferred embodiment, the control level is established from control samples that are from patients or cell lines known to be resistant or sensitive to EGFR inhibitor. In one aspect, the control samples were obtained from a population of matched individuals. According to the present invention, the phrase “matched individuals” refers to a matching of the control individuals on the basis of one or more characteristics which are suitable for the type of cell or tumor growth to be evaluated. For example, control individuals can be matched with the patient to be evaluated on the basis of gender, age, race, or any relevant biological or sociological factor that may affect the baseline of the control individuals and the patient (e.g., preexisting conditions, consumption of particular substances, levels of other biological or physiological factors). To establish a control level, samples from a number of matched individuals are obtained and evaluated in the same manner as for the test samples. The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test patient). The values obtained from the control samples are statistically processed using any suitable method of statistical analysis to establish a suitable baseline level using methods standard in the art for establishing such values.

It will be appreciated by those of skill in the art that a control level need not be established for each assay as the assay is performed but rather, a baseline or control can be established by referring to a form of stored information regarding a previously determined control level for sensitive and resistant patients (responders and non-responders), such as a control level established by any of the above-described methods. Such a form of stored information can include, for example, but is not limited to, a reference chart, listing or electronic file of population or individual data regarding sensitive and resistant tumors/patients, or any other source of data regarding control level gene amplification or polysomy that is useful for the patient to be evaluated.

The method of the present invention includes the use of EGFR inhibitors, HDAC inhibitors, or an agonist thereof, or a drug having substantially similar biological activity as the EGFR inhibitor or HDAC inhibitor. An agonist, as used herein, is a compound that is characterized by the ability to agonize (e.g., stimulate, induce, increase, enhance, or mimic) the biological activity of a naturally occurring or reference protein or compound. More particularly, an agonist can include, but is not limited to, a compound, protein, peptide, or nucleic acid that mimics or enhances the activity of the natural or reference compound, and includes any homologue, mimetic, or any suitable product of drug/compound/peptide design or selection which is characterized by its ability to agonize (e.g., stimulate, induce, increase, enhance) the biological activity of a naturally occurring or reference compound. In contrast, an antagonist refers to any compound which inhibits (e.g., antagonizes, reduces, decreases, blocks, reverses, or alters) the effect of a naturally occurring or reference compound as described above. More particularly, an antagonist is capable of acting in a manner relative to the activity of the reference compound, such that the biological activity of the natural or reference compound, is decreased in a manner that is antagonistic (e.g., against, a reversal of, contrary to) to the natural action of the reference compound. Such antagonists can include, but are not limited to, any compound, protein, peptide, or nucleic acid (including ribozymes and antisense) or product of drug/compound/peptide design or selection that provides the antagonistic effect.

Agonists and antagonists that are products of drug design can be produced using various methods known in the art. Various methods of drug design, useful to design mimetics or other compounds useful in the present invention are disclosed in Maulik et al., 1997, Molecular Biotechnology: Therapeutic Applications and Strategies, Wiley-Liss, Inc., which is incorporated herein by reference in its entirety. An agonist or antagonist can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, natural or synthetic steroidal compounds, carbohydrates and/or natural or synthetic organic and non-steroidal molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

A drug having substantially similar biological activity as an HDAC inhibitor or an EGFR inhibitor described herein refers to a drug having substantially any function(s) exhibited or performed by the reference compound that is ascribed to the reference compound as measured or observed in vivo (i.e., under physiological conditions) or in vitro (i.e., under laboratory conditions).

Another embodiment of the invention includes an assay kit comprising: (a) a means for detecting a level of a biomarker or a combination of biomarkers selected from: a level of expression of E-cadherin; and/or a level of expression of a component of TF8, preferably ZEB1; and (b) information containing a predetermined control level of E-cadherin transcripts and/or protein; and/or information containing a predetermined control level of a component of TF8 transcripts and/or protein, preferably ZEB1. The kit can further include a means for detecting a level of a biomarker or combination of biomarkers selected from: (i) a level of amplification of the epidermal growth factor receptor (EGFR) gene; (ii) a level of polysomy of the EGFR gene; (iii) a level of amplification of the human tyrosine kinase receptor-type receptor (HER2) gene; (iv) a level of polysomy of the HER2 gene; (v) a level of EGFR protein expression; (vi) a level of phosphorylated Akt protein expression. Appropriate controls would also be included.

In one embodiment, a means for detecting E-cadherin, or a component of TF8, or for detecting EGFR or HER2 genes or proteins or other biomarkers, can generally be any type of reagent that can be used in a method of the present invention. Such a means for detecting include, but are not limited to: a probe that hybridizes under stringent hybridization conditions to a gene (e.g., an EGFR gene), antibodies reactive to E-cadherin peptides or a component of TF8 peptides, and labeled probes that hybridize to E-cadherin transcripts or a component of TF8 RNA transcripts. Nucleic acid sequences and protein sequences for these genes and proteins are known in the art and can be used to produce such reagents for detection.

The means for detecting of the assay kit of the present invention can be conjugated to a detectable tag or detectable label. Such a tag can be any suitable tag which allows for detection of the reagents used to detect the gene of interest and includes, but is not limited to, any composition or label detectable by spectroscopic, photochemical, electrical, optical or chemical means. Useful labels in the present invention include fluorescent dyes (e.g., fluorescein, texas red, rhodamine, green fluorescent protein, and the like), radiolabels (e.g., ³H, ¹²⁵I, ³⁵S, ¹⁴C, or ³²P), and colorimetric labels.

In addition, the means for detecting of the assay kit of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly effecting the activity and/or ability of the detection means to detect the desired target molecule. Exemplary organic solid supports include polymers such as polystyrene, nylon, phenol-formaldehyde resins, and acrylic copolymers (e.g., polyacrylamide).

The kits of the invention can further include predetermined instructions for administration of the combination therapy of an EGFR inhibitor and an HDAC inhibitor of the invention, and in some embodiments, may further include doses of an EGFR inhibitor and/or an HDAC inhibitor to administer to a patient.

The Examples, which follow, are illustrative of specific embodiments of the invention, and various uses thereof. They are set forth for explanatory purposes only, and are not to be taken as limiting the invention.

### EXAMPLES

The following materials and methods were used in all Examples presented herein.

Materials and Methods

Cell Culture, Drugs and MTS assay. Twenty NSCLC cell lines were used: squamous (NCI-H157, HCC95, HCC15 and H441), large-cell (H460, H1299, H2126 and H1264, a derivative of H460), adeno (Calu3, A549, H2122, H1648, H520, HCC78, HCC193, H2009, HCC44 and H3255) and bronchioalveolar (H358 and H322). The NSCLC cell lines, HCC78, H2126, HCC95, H1299, HCC193, HCC44, HCC15, H2009 were obtained from UTSW and the H3255 was a gift from Dr. Bruce Johnson. All lines were cultured in RPMI medium 1640 under standard conditions. Gefitinib was a gift of AstraZeneca, MS-275 was a gift from Nihon Schering K.K. Stock solutions were prepared in dimethyl sulfoxide and stored at −20° C. The drugs were diluted in fresh media before each experiment, and the final dimethyl sulfoxide concentration was <0.1%. Epidermal growth factor (EGF) was purchased from R&D Systems Inc. (Minneapolis, Minn.). Growth inhibition was assessed by MTS (3-(4,5-dimethylthiazol-2-yl)-5-(3-carboxymethoxyphenyl)-2-(4-sulfophenyl)2H-tetrazolium, inner salt) assay (Promega, Madison, Wis.). Briefly, 2.103 NSCLC cells are plated in each well of 96-well flat-bottomed microtiter plates. Gefitinib was added when cell cultures became 50-80% confluent. After 4 day incubation, 50 μl of a 2 mg/ml solution of the tetrazolium salt MTT (Promega), dissolved in RPMI 1640, is added to each well. The microtiter plates were incubated for 4 h at 37° C. The absorbency of each well is measured using an automated plate reader. The data was analyzed using a SlideWrite program to determine the IC₅₀ of the drug. Cell Lysis and Western Blots and immunohistochemistry. Cells were disrupted in lysis buffer (10 mM Tris-HCl, pH 7.5/150 mM NaCl/0.5% IGEPAL/0.5 mM PMSF/10 μg/ml leupeptin/5 μg/ml pepstatin A/2.1 μg/ml aprotinin) on ice. After sonication, the Bradford assay was used for protein quantification. Protein lysates (30-50 μg) were separated by gel electrophoresis on 7.5%-10% polyacrylamide and analyzed by Western blot using PVDF membranes (Bio-Rad Laboratories, Inc., Richmond, Calif.). Anti-EGFR and the phospho-specific EGFR (pY1068), (Cell Signaling, Beverly, Mass.) were used at 1:1,000. E-cad and β Actin antibodies (BD Biosciences Pharmingen/Transduction Laboratories, San Jose, Calif.; Sigma-Aldrich, #A5316, Saint Louis, Miss.) were used at 1:3,000, 1:5000 dilutions, respectively. Detection used horseradish peroxidase-conjugated secondary antibodies and chemiluminescence (Amersham Biosciences, Inc.). The anti-E-cad antibody reacting with the cytoplasmic domain of the molecule (mouse monoclonal, clone 36, Transduction Laboratories, Lexington, Ky.) was applied at 1/100 dilution to sectioned paraffin-embedded cell lines. Antigen retrieval was performed in citrate buffer using a Biocare Medical (Walnut Creek, Calif.) decloaking chamber. Peroxide blocking was performed with 3% peroxide in absolute methanol. Blocking was performed with Powerblock (Biogenics, San Ramon, Calif.) or avidin/biotin block. After incubation of primary antibodies for 1 hour at 37° C. the secondary antibody (Dako Biotinylated Multi-Link antimouse, immunoglobulin with 40% human serum) was applied for 30 minutes at room temperature. This was followed by application of streptavidin horseradish peroxidase enzyme complex and diaminobenzidine chromogen. The slides were then counterstained in hematoxylin and covered with a coverslip.

RNA, Primers, and Quantitative Real-Time RT-PCR. Total RNA was prepared from NSCLC cell lines using the RNAeasy (Qiagen). During the preparation all samples were treated with RNase-free DNase 1 (10 mg/ml, Qiagen) prior to cDNA synthesis. cDNA was synthesized as part of the RT-PCR reaction from 0.3 mg total RNA. Quantitative Real-Time RT-PCR assays were performed using the SYBR Green RT-PCR Kit (Qiagen) using a GeneAmp 5700 Sequence Detector (Applied Biosystems), which allows amplification and detection (by fluorescence) in the same tube, using a kinetic approach. Amplification data were analyzed by using GENEAMP 5700 SDS software, converted into cycle numbers at a set cycle threshold (Ct values) and quantified in relation to a standard. Human adult-lung (Clontech Lab. Inc) or human fetal-lung RNA (Stratagene) was used as standards in all the experiments. Standards were used at 20, 100, 500 mg. In each experiment a no-template control was used as a control. To normalize for the amount of input cDNA, the quantified relative amount of the generated product was divided by the amount generated for the housekeeping gene beta-Actin. All samples were performed in triplicates.

Cell Cycle Analysis. NSCLC Cells were plated at a density of 0.5×10⁶ cells/well in 6 well plates. Gefitinib was added to the medium after 24 hours, and the cells were incubated for another 72 hours, after which the cells were analyzed as described previously. The percentage of apoptosis was estimated from the sub-G₁ cell fraction.

### Example 1

The following example describes E-cad expression in gefitinib-sensitive and gefitinib-resistant NSCLC cell lines.

A set of 21 NSCLC and one uterine cell line using the MTT assay were analyzed for their growth inhibition by gefitinib. Of the 21 NSCLC, six cell lines H3255, H358, H322, Calu3, H1648, HCC78 had IC₅₀ of <1 μM, whereas six cell lines HCC15, H157, H460, H520, and H1264 (a duplicate cell line of H460) had IC₅₀ of ≧10 μM. This diverse growth response to gefitinib was used to identify genes differentially expressed in this set of cell lines.

Using real-time RT-PCR, a positive correlation was detected between the expression of E-cad and sensitivity to gefitinib (r=0.76, p0.0001). The highest E-cad expression was detected in the most sensitive cell line, H3255 (IC₅₀=0.015 μM) that harbors the EGFR mutation L858R. This positive correlation was detected in E-cad expression in microarrays developed from the 20 cell lines (r=0.74, p=0.0002). At the protein level, expression of E-cad was evaluated in 11 NSCLC cell lines western blot analysis. As shown previously, there was no correlation between EGFR expression and sensitivity to gefitinib. However, there was 100% correlation between presence or absence of E-cad expression and sensitivity or resistance to gefitinib, respectively.

Using immunohistochemistry, the expression of E-cadherin was also evaluated in two cell lines sensitive to (A431 and Calu3), and two cell lines resistant to gefitinib (H520 and H157). In the sensitive cell lines, strong expression of E-cad was detected with membranous and cytoplasmic localization, whereas expression was absent in the two resistant cell lines.

### Example 2

The following example describes the expression of E-cad regulatory molecules in NSCLC cell lines.

It is known that there is involvement of the Wnt pathway in regulating E-cad expression. The expression of molecules in the Wnt/E-cad pathway (Wnt1, Wnt5A, Wnt5B, Wnt6, Wnt7A, frizzled, axin1, disheveled, GSK3, α-catenin, β-catenin, γ-catenin and E-cad) were screened in the Affimetrix data of microarrays of cell lines with IC₅₀<10 μM (H3255, H358, H322, Calu3, H1648, HCC78) and with IC₅₀>10 μM (H157, H520, H460 and H1264). E-cad had the highest fold upregulation in the sensitive cell lines compared to the resistant cell lines (200 fold). None of the other molecules in the wnt pathway had similar differential expression between the sensitive and resistant cell lines.

E-cad regulation involves four zinc finger transcription factors TF-8, slug, snail and SIP1. Evaluation of the cell lines microarray data revealed that TF-8 had the highest difference in expression between the sensitive and resistant cell lines (10.4 fold) compared to the other three molecules, SIP1, snail, and slug.

The expression of TF-8 was confirmed using RT-PCR. A negative correlation was detected between TF-8 expression and sensitivity to gefitinib in the 20 NSCLC cell lines (r=−0.74, p=0.0002). This negative correlation between TF-8 expression and gefitinib-sensitivity was detected in microarrays developed from the 20 cell lines (r=0.71, p=0.0004).

### Example 3

The following example describes the effect of E-cadherin on gefitinib induced apoptosis in NSCLC cell lines.

The effect of gefitinib on inducing apoptosis and cell death in NSCLC cell lines sensitive and resistant to gefitinib was evaluated. When cell lines were treated with 10 μM of gefitinib a 35 fold increase in apoptosis and cell death was detected in the most sensitive cell line H3255. At the same concentration there was a 2.3-3.4 fold increase in apoptosis and cell death in the less sensitive cell lines (H322, H358 and Calu3), whereas, no apoptotic or necrotic effect was detected in the more resistant cell lines (H460, H520, H157 and A549).

The effect of E-cad on NSCLC cell lines apoptotic response to gefitinib was assessed by transfecting a gefitinib-resistant cell line, H157, with an E-cad-encoding adenovirus. This cell line was selected for its lack expression of E-cad, the presence of EGFR and its resistance to gefitinib. The H157 cell line was transfected with E-cad and two stable transfected lines were developed, H157-E-cad-3 and H157-E-cad-8. H157 cell line transfected with a GFP construct was used as control. Expression of E-cad was verified by western blot. Higher expression of E-cad was detected in the H157-E-cad-3 cell line compared to the H157-E-cad-3 cell line. Previous studies indicated the interaction between EGFR and E-cad. We evaluated the effect of the ectopic expression of E-cad on EGFR phosphorylation and response to EGF. Ectopic expression of E-cad did not lead to EGFR activation (phosphorylation). However, two fold increase in phosphorylation was detected in transfected cell lines treated with EGF.

The effect of the ectopic expression of E-cad on cell survival was evaluated. Three and nine fold increased in ratio of apoptotic to viable cells was detected in both the cell lines, H157-Ecad-8 and H157-Ecad-3 (8.8:87.8% to 21:69% and 43.5:48.4%, respectively) as compared to the control cell line H157-GFP. Response to gefitinib was further enhanced. Cell lines were treated with 10 μM of gefitinib for 48 hours and apoptosis and necrosis was evaluated using annexin V and propidium iodine. Six and thirteen fold increase in ratio apoptotic to viable cells (8.4:87.4% to 31.5:55.3%; 8.4:87.4 to 49.8:37.8%, respectively) and three to nine fold increase ratio necrotic to viable cells (11.5:88.1 to 26.1:70.6; 11.5:88.1 to 52.9:45.8) was detected in the H157-E-cad-3 and H157-E-cad-8 cell line compared to the control cell line H157-GFP when treated with gefitinib.

These data indicate that restoring E-cad expression lead to an increase in apoptosis and it restores the effect of gefitinib on cell lines resistant to gefitinib.

### Example 4

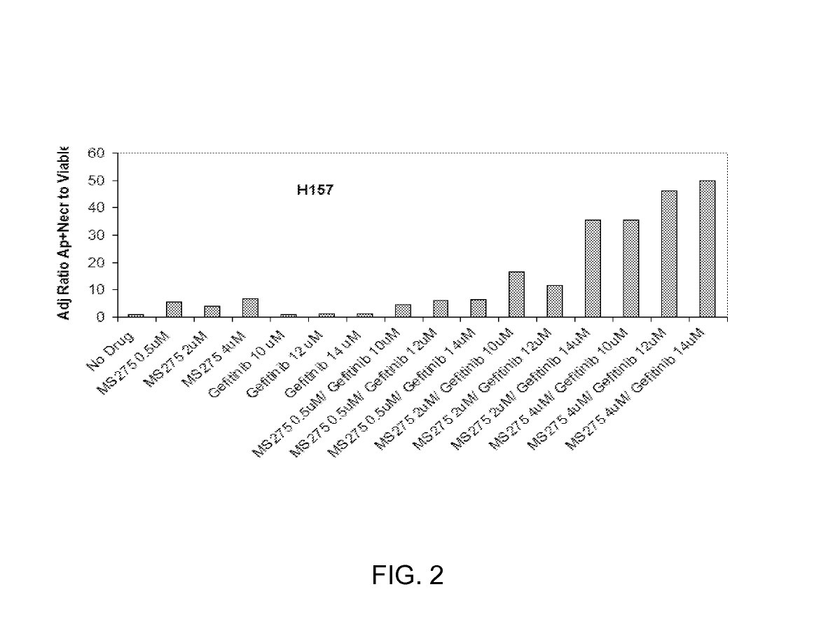

The following example shows that histone deacetylase HDAC inhibitors reverse resistance to gefitinib. It is known that E-cadherin expression is restored in NSCLC by inhibiting HDAC with TSA. The inventors determined whether pretreatment of NSCLC cell lines with HDACi will lead to changes in gene and protein expression and improve sensitivity to gefitinib. The IC of MS-275 was evaluated in the gefitinib-resistant NSCLC cell lines H157, H520, and H460. The IC₂₅₋₇₅ in these cell lines was detected between 0.5 and 4 M. Expression of E-cad was evaluated in these cell lines. Eight to twelve fold upregulation of E-cad expression was detected in all the cell lines tested 24 hours after treatment with 4 or 10 μM MS-275. Next the inventors evaluated the effect of pretreatment of the NSCLC lung cancer cell lines with MS-275 on their response to gefitinib. The NSCLC cell lines H157, H520, H460, and H1703 were treated with the HDAC inhibitor, MS-275 alone, with gefitinib alone or with MS-275, 24 hours prior to treatment with gefitinib. A synergistic effect was detected by the sequential use of MS-275 followed by gefitinib in these cell lines. Increasing doses of MS-275 are used. Cell death was several folds higher when cell lines were treated sequentially with the two drugs, compared to treatment with each drug alone. See FIG. 2, showing the effect of treatment with either gefitinib alone or with combination therapy of gefitinib and MS-275, on H175 cells' adjusted ratio of apoptotic and necrotic cells to viable cells.

### Example 5

The following example is a method to identify histone deacetylase HDAC inhibitors (HDACi) that reverse resistance to EGFR inhibitors in colorectal cancer cell lines (CRC cell line). To evaluate the effect of pretreatment of the CRC cell lines with MS-275 (HDAC inhibitor) on their response to gefitinib (EGFR inhibitor), the CRC cell lines HCT-116HCT-115, and SW480 were treated with the MS-275 alone, with gefitinib alone or with a combination of both MS-275 and gefitinib. Cell apoptosis and cell death in the cell lines was then analyzed as described previously for NSCLC cell lines: The percentage of apoptosis in the HCT-116 cell line was estimated from the sub-G₁ cell fraction and the necrotic cell population. A normalized ratio of apoptotic+necrotic cells versus live cells showed a synergistic effect for the combination of MS-275 and gefitinib over treatment with either compound alone. HCT-116 cells had a ratio 3 times greater than gefitinib alone, and a ratio 2 times greater than MS-275 alone.

### Example 6

The following example is a method to identify histone deacetylase HDAC inhibitors (HDACi) that reverse resistance to EGFR inhibitors in breast cancer cell lines. To evaluate the effect of pretreatment of the breast cancer cell lines with MS-275 (HDAC inhibitor) on their response to erlotinib (EGFR inhibitor), the breast cancer cell lines are treated with the MS-275 alone, with erlotinib alone or with a combination of both MS-275 and erlotinib. Cell apoptosis and cell death in the breast cancer cell lines is then analyzed as described previously for NSCLC and CRC cell lines: The percentage of apoptosis in the breast cancer cell lines is estimated from the sub-G1 cell fraction and the necrotic cell population. A normalized ratio of apoptotic+necrotic cells versus live cells shows the effect for the combination of MS-275 and erlotinib over treatment with either compound alone.

### Example 7

The following example is a method to identify histone deacetylase HDAC inhibitors (HDACi) that reverse resistance to EGFR inhibitors in breast cancer cell lines. To evaluate the effect of pretreatment of the breast cancer cell lines with MS-275 (HDAC inhibitor) on their response to lapatinib (Tykerb®, an EGFR/HER2 inhibitor), the breast cancer cell lines are treated with the MS-275 alone, with lapatinib alone or with a combination of both MS-275 and lapatinib. Cell apoptosis and cell death in the breast cancer cell lines is then analyzed as described previously for NSCLC and CRC cell lines: The percentage of apoptosis in the breast cancer cell lines is estimated from the sub-G1 cell fraction and the necrotic cell population. A normalized ratio of apoptotic+necrotic cells versus live cells shows the effect for the combination of MS-275 and lapatinib over treatment with either compound alone.

### Example 8

The following is an example the evaluation of the synergistic effect of HDAC inhibitors in combination with EGFR inhibitors in colorectal carcinoma (CRC) in vivo. The activity of an HDAC inhibitor as single agent and in combination with EGFR inhibitor is evaluated in nude mice bearing CRC cell lines. Mice bearing CRC tumors are randomly assigned to treatment groups, and the effect of MS-275, erlotinib, and an MS-275/erlotinib combination on tumor growth is evaluated. Nude mice are implanted with EGFR sensitive or EGFR resistant CRC cell-lines. Resistance and sensitivity of the cell line is determined as described previously herein. Implantation of a tumor is achieved through established tumor transplantation techniques (e.g., injection or surgical orthotopic implantation). Upon establishment of the CRC tumor, as determined by tumor volume measurement, the effect of MS-275, erlotinib, and a combination of MS-275 and erlotinib is evaluated for inhibition of tumor growth. Each compound (MS-275, erlotinib, or MS-275/erlotinib combination) is administered to different groups of mice in different dosages. Each compound is administered as follows: MS-275-2 doses, erlotinib—2 doses, MS-275/erlotinib combination—4 doses. Biopsies and measurements of the tumors are taken at 4 time points corresponding to 0, 48, 72, and 96 hours post-treatment. Tumor volumes are measured for each time point to determine efficacy of the compounds. Expression of E-cadherin, Erb3, EGFR, and Her3 are determined by RT-PCR from the biopsies. Expression of E-cadherin, Her3, EGFR, cyclin D1, and cyclin D3 are detected in biopsied tissue by standard immunohistochemistry techniques. The effect of MS_{—}275 on EGFR sensitive and resistant cancers is determined by evaluation of tumor growth. The effect of MS-275 and erlotinib on the expression of E-cadherin, Erb3, Her3, EGFR, cyclin D1, and cyclin D3 is determined and correlated with CRC sensitivity or resistance to MS-275/erlotinib combination therapy.

### Example 9

The following is an example the evaluation of the synergistic effect of HDAC inhibitors in combination with EGFR inhibitors in breast cancer in vivo. The activity of an HDAC inhibitor as single agent and in combination with EGFR inhibitor is evaluated in nude mice bearing breast cancer cell lines. Mice bearing breast cancer tumors are randomly assigned to treatment groups, and the effect of MS-275, erlotinib, and an MS-275/erlotinib combination on tumor growth is evaluated. Nude mice are implanted with EGFR sensitive or EGFR resistant breast cancer cell lines. Resistance and sensitivity of the cell line is determined as described previously herein Implantation of a tumor is achieved through established tumor transplantation techniques (e.g., injection or surgical orthotopic implantation). Upon establishment of the breast cancer tumor, as determined by tumor volume measurement, the effect of MS-275, erlotinib, and a combination of MS-275 and erlotinib is evaluated for inhibition of tumor growth. Each compound (MS-275, erlotinib, or MS-275/erlotinib combination) is administered to different groups of mice in different dosages. Each compound is administered as follows: MS-275—2 doses, erlotinib—2 doses, MS-275/erlotinib combination—4 doses. Biopsies and measurements of the tumors are taken at 4 time points corresponding to 0, 48, 72, and 96 hours post-treatment. Tumor volumes are measured for each time point to determine efficacy of the compounds. Expression of E-cadherin, Erb3, EGFR, and Her3 are determined by RT-PCR from the biopsies. Expression of E-cadherin, Her3, EGFR, cyclin D1, and cyclin D3 are detected in biopsied tissue by standard immunohistochemistry techniques. The effect of MS-275 on EGFR sensitive and resistant cancers is determined by evaluation of tumor growth. The effect of MS-275 and erlotinib on the expression of E-cadherin, Erb3, Her3, EGFR, cyclin D1, and cyclin D3 is determined and correlated with breast cancer tumor sensitivity or resistance to MS-275/erlotinib combination therapy.

### Example 10

Human Clinical Trial of the Safety and/or Efficacy of MS-275/gefitinib combination therapy

Objective: To compare the safety and pharmacokinetics of administered MS-275 and gefitinib.

Study Design This will be a Phase I, single-center, open-label, randomized dose escalation study followed by a Phase II study in EGFR inhibitor-resistant and EGFR inhibitor-sensitive metastatic cancer patients with disease that can be biopsied (i.e., breast cancer, non-small cell lung cancer, prostate cancer, pancreatic cancer, colorectal cancer, head and neck cancer). Patients should not have had exposure to MS-275 or gefitinib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral gefitinib daily and oral MS-275 on days 3 and 10. Treatment repeats every 28 days in the absence of unacceptable toxicity. Cohorts of 3-6 patients receive escalating doses of gefitinib and MS-275 until the maximum tolerated dose (MTD) for the combination of gefitinib and MS-275 is determined. Test dose ranges are initially determined via the established individual dose ranges for MS-275 and gefitinib. A standard dosage for gefitinib is 1250 mg/daily. An established dosage for MS-275 includes 2 mg/m² weekly. Additional dosages, both decreasing and increasing in amount as well a frequency, are determined based on the standard dose for both MS-275 and gefitinib. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive gefitinib as in phase I at the MTD determined in phase I and MS-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling Serial blood is drawn by direct vein puncture before and after administration of MS-275 or gefitinib. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of MS-275 and gefitinib will be safe and well tolerated by cancer patients. The combination of MS-275 and gefitinib provides large clinical utility to cancer patients.

### Example 11

Human Clinical Trial of the Safety and/or Efficacy of MS-275/lapatinib (Tykerb®) combination therapy for breast cancer.

Objective: To compare the safety and pharmacokinetics of administered MS-275 and lapatinib (Tykerb®) in breast cancer patients.

Study Design This will be a Phase I, single-center, open-label, randomized dose escalation study followed by a Phase II study in EGFR inhibitor-resistant and EGFR inhibitor-sensitive breast cancer patients with disease that can be biopsied. Patients should not have had exposure to MS-275 or lapatinib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral lapatinib daily and oral MS-275 on days 3 and 10. Treatment repeats every 28 days in the absence of unacceptable toxicity. Cohorts of 3-6 patients receive escalating doses of lapatinib and MS-275 until the maximum tolerated dose (MTD) for the combination of lapatinib and MS-275 is determined. Test dose ranges are initially determined via the established individual dose ranges for MS-275 and lapatinib. A standard dosage for lapatinib is 1250 mg/daily. An established dosage for MS-275 includes 2 mg/m² weekly. Additional dosages, both decreasing and increasing in amount as well a frequency, are determined based on the standard dose for both MS-275 and lapatinib. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive lapatinib as in phase I at the MTD determined in phase I and MS-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria

Blood Sampling Serial blood is drawn by direct vein puncture before and after administration of MS-275 or lapatinib. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated. Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon feasibility/availability, and the same imaging modality is utilized throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of MS-275 and lapatinib will be safe and well tolerated by breast cancer patients. The combination of MS-275 and lapatinib provides large clinical utility to breast cancer patients.

### Example 12

Human Clinical Trial of the Safety and/or Efficacy of MS-275/erlotinib combination therapy for colorectal cancer.

Objective: To compare the safety and pharmacokinetics of administered MS-275 and erlotinib in colorectal cancer patients.

Study Design This will be a Phase I, single-center, open-label, randomized dose escalation study followed by a Phase II study in EGFR inhibitor-resistant and EGFR inhibitor-sensitive colorectal cancer patients with disease that can be biopsied. Patients should not have had exposure to MS-275 or erlotinib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral erlotinib daily and oral MS-275 on days 3 and 10. Treatment repeats every 28 days in the absence of unacceptable toxicity. Cohorts of 3-6 patients receive escalating doses of erlotinib and MS-275 until the maximum tolerated dose (MTD) for the combination of erlotinib and MS-275 is determined. Test dose ranges are initially determined via the established individual dose ranges for MS-275 and erlotinib. A standard dosage for erlotinib is 1250 mg/daily. An established dosage for MS-275 includes 2 mg/m² weekly. Additional dosages, both decreasing and increasing in amount as well a frequency, are determined based on the standard dose for both MS-275 and erlotinib. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive erlotinib as in phase I at the MTD determined in phase I and MS-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria

Blood Sampling Serial blood is drawn by direct vein puncture before and after administration of MS-275 or erlotinib. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon feasibility/availability, and the same imaging modality is utilized throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of MS-275 and erlotinib will be safe and well tolerated by colorectal cancer patients. The combination of MS-275 and erlotinib provides large clinical utility to colorectal cancer patients.

### Example 13

Human Clinical Trial of the Safety and/or Efficacy of MS-275/lapatinib (Tykerb®) combination therapy for bladder cancer.

Objective: To compare the safety and pharmacokinetics of administered MS-275 and lapatinib (Tykerb®) in bladder cancer patients.

Study Design This will be a Phase I, single-center, open-label, randomized dose escalation study followed by a Phase II study in EGFR inhibitor-resistant and EGFR inhibitor-sensitive bladder cancer patients with disease that can be biopsied. Patients should not have had exposure to MS-275 or lapatinib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral lapatinib daily and oral MS-275 on days 3 and 10. Treatment repeats every 28 days in the absence of unacceptable toxicity. Cohorts of 3-6 patients receive escalating doses of lapatinib and MS-275 until the maximum tolerated dose (MTD) for the combination of lapatinib and MS-275 is determined. Test dose ranges are initially determined via the established individual dose ranges for MS-275 and lapatinib. A standard dosage for lapatinib is 1250 mg/daily. An established dosage for MS-275 includes 2 mg/m² weekly. Additional dosages, both decreasing and increasing in amount as well a frequency, are determined based on the standard dose for both MS-275 and lapatinib. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive lapatinib as in phase I at the MTD determined in phase I and MS-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria

Blood Sampling Serial blood is drawn by direct vein puncture before and after administration of MS-275 or lapatinib. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon feasibility/availability, and the same imaging modality is utilized throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of MS-275 and lapatinib will be safe and well tolerated by bladder cancer patients. The combination of MS-275 and lapatinib provides large clinical utility to bladder cancer patients.

### Example 14

Human Clinical Trial of the Safety and/or Efficacy of MS-275/erlotinib combination therapy for non-small cell lung cancer.

Objective: To compare the safety and pharmacokinetics of administered MS-275 and erlotinib in non-small cell lung cancer patients.

Study Design This will be a Phase I, single-center, open-label, randomized dose escalation study followed by a Phase II study in EGFR inhibitor-resistant and EGFR inhibitor-sensitive non-small cell lung cancer patients with disease that can be biopsied. Patients should not have had exposure to MS-275 or erlotinib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral erlotinib daily and oral MS-275 on days 3 and 10. Treatment repeats every 28 days in the absence of unacceptable toxicity. Cohorts of 3-6 patients receive escalating doses of erlotinib and MS-275 until the maximum tolerated dose (MTD) for the combination of erlotinib and MS-275 is determined. Test dose ranges are initially determined via the established individual dose ranges for MS-275 and erlotinib. A standard dosage for erlotinib is 1250 mg/daily. An established dosage for MS-275 includes 2 mg/m² weekly. Additional dosages, both decreasing and increasing in amount as well a frequency, are determined based on the standard dose for both MS-275 and erlotinib. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive erlotinib as in phase I at the MTD determined in phase I and MS-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria

Blood Sampling Serial blood is drawn by direct vein puncture before and after administration of MS-275 or erlotinib. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon feasibility/availability, and the same imaging modality is utilized throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of MS-275 and erlotinib will be safe and well tolerated by non-small cell lung cancer patients. The combination of MS-275 and erlotinib provides large clinical utility to non-small cell lung cancer patients.

### Example 15

Human Clinical Trial of the Safety and/or Efficacy of Scriptaid (S7817 Sigma)/erlotinib combination therapy for non-small cell lung cancer.

Objective: To compare the safety and pharmacokinetics of administered Scriptaid and erlotinib in non-small cell lung cancer patients.

Study Design This will be a Phase I, single-center, open-label, randomized dose escalation study followed by a Phase II study in EGFR inhibitor-resistant and EGFR inhibitor-sensitive non-small cell lung cancer patients with disease that can be biopsied. Patients should not have had exposure to Scriptaid or erlotinib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral erlotinib daily and oral Scriptaid on days 3 and 10. Treatment repeats every 28 days in the absence of unacceptable toxicity. Cohorts of 3-6 patients receive escalating doses of erlotinib and Scriptaid until the maximum tolerated dose (MTD) for the combination of erlotinib and Scriptaid is determined. Test dose ranges are initially determined via the established individual dose ranges for Scriptaid and erlotinib. A standard dosage for erlotinib is 1250 mg/daily. An established dosage for Scriptaid includes 2 mg/m² weekly. Additional dosages, both decreasing and increasing in amount as well a frequency, are determined based on the standard dose for both Scriptaid and erlotinib. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive erlotinib as in phase I at the MTD determined in phase I and Scriptaid as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria

Blood Sampling Serial blood is drawn by direct vein puncture before and after administration of Scriptaid or erlotinib. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon feasibility/availability, and the same imaging modality is utilized throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of Scriptaid and erlotinib will be safe and well tolerated by non-small cell lung cancer patients. The combination of Scriptaid and erlotinib provides large clinical utility to non-small cell lung cancer patients.

### Example 16

Human Clinical Trial of the Safety and/or Efficacy of MS-275/Sutent combination therapy for non-small cell lung cancer.

Objective: To compare the safety and pharmacokinetics of administered MS-275 and sutent in non-small cell lung cancer patients.

Study Design This will be a Phase I, single-center, open-label, randomized dose escalation study followed by a Phase II study in EGFR inhibitor-resistant and EGFR inhibitor-sensitive non-small cell lung cancer patients with disease that can be biopsied. Patients should not have had exposure to MS-275 or sutent prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral sutent daily and oral MS-275 on days 3 and 10. Treatment repeats every 28 days in the absence of unacceptable toxicity. Cohorts of 3-6 patients receive escalating doses of sutent and MS-275 until the maximum tolerated dose (MTD) for the combination of sutent and MS-275 is determined. Test dose ranges are initially determined via the established individual dose ranges for MS-275 and sutent. A standard dosage for sutent is 50 mg/daily. An established dosage for MS-275 includes 2 mg/m² weekly. Additional dosages, both decreasing and increasing in amount as well a frequency, are determined based on the standard dose for both MS-275 and sutent. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive sutent as in phase I at the MTD determined in phase I and MS-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria

Blood Sampling Serial blood is drawn by direct vein puncture before and after administration of MS-275 or sutent. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon feasibility/availability, and the same imaging modality is utilized throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of MS-275 and sutent will be safe and well tolerated by non-small cell lung cancer patients. The combination of MS-275 and sutent provides large clinical utility to non-small cell lung cancer patients.

### Example 17

Human Clinical Trial of the Safety and/or Efficacy of MS-275/erlotinib combination therapy for pancreatic cancer.

Objective: To compare the safety and pharmacokinetics of administered MS-275 and erlotinib in pancreatic cancer patients.

Study Design This will be a Phase I, single-center, open-label, randomized dose escalation study followed by a Phase II study in EGFR inhibitor-resistant and EGFR inhibitor-sensitive pancreatic cancer patients with disease that can be biopsied. Patients should not have had exposure to MS-275 or erlotinib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral erlotinib daily and oral MS-275 on days 3 and 10. Treatment repeats every 28 days in the absence of unacceptable toxicity. Cohorts of 3-6 patients receive escalating doses of erlotinib and MS-275 until the maximum tolerated dose (MTD) for the combination of erlotinib and MS-275 is determined. Test dose ranges are initially determined via the established individual dose ranges for MS-275 and erlotinib. A standard dosage for erlotinib is 1250 mg/daily. An established dosage for MS-275 includes 2 mg/m² weekly. Additional dosages, both decreasing and increasing in amount as well a frequency, are determined based on the standard dose for both MS-275 and erlotinib. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive erlotinib as in phase I at the MTD determined in phase I and MS-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria

Blood Sampling Serial blood is drawn by direct vein puncture before and after administration of MS-275 or erlotinib. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon feasibility/availability, and the same imaging modality is utilized throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of MS-275 and erlotinib will be safe and well tolerated by pancreatic cancer patients. The combination of MS-275 and erlotinib provides large clinical utility to pancreatic cancer patients.

### Example 18

Human Clinical Trial of the Safety and/or Efficacy of SAHA/erlotinib combination therapy for pancreatic cancer.

Objective: To compare the safety and pharmacokinetics of administered SAHA and erlotinib in pancreatic cancer patients.

Study Design This will be a Phase I, single-center, open-label, randomized dose escalation study followed by a Phase II study in EGFR inhibitor-resistant and EGFR inhibitor-sensitive pancreatic cancer patients with disease that can be biopsied. Patients should not have had exposure to SAHA or erlotinib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral erlotinib daily and oral SAHA on days 3 and 10. Treatment repeats every 28 days in the absence of unacceptable toxicity. Cohorts of 3-6 patients receive escalating doses of erlotinib and SAHA until the maximum tolerated dose (MTD) for the combination of erlotinib and SAHA is determined. Test dose ranges are initially determined via the established individual dose ranges for SAHA and erlotinib. A standard dosage for erlotinib is 1250 mg/daily. An established dosage for SAHA includes 2 mg/m² weekly. Additional dosages, both decreasing and increasing in amount as well a frequency, are determined based on the standard dose for both SAHA and erlotinib. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive erlotinib as in phase I at the MTD determined in phase I and SAHA as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria

Blood Sampling Serial blood is drawn by direct vein puncture before and after administration of SAHA or erlotinib. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon feasibility/availability, and the same imaging modality is utilized throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of SAHA 5 and erlotinib will be safe and well tolerated by pancreatic cancer patients. The combination of SAHA and erlotinib provides large clinical utility to pancreatic cancer patients.

### Example 19

Human Clinical Trial of the Safety and/or Efficacy of MS-275/Sutent combination therapy for renal cancer.

Objective: To compare the safety and pharmacokinetics of administered MS-275 and Sutent in renal cancer patients.

Study Design This will be a Phase I, single-center, open-label, randomized dose escalation study followed by a Phase II study in EGFR inhibitor-resistant and EGFR inhibitor-sensitive renal cancer patients with disease that can be biopsied. Patients should not have had exposure to MS-275 or Sutent prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral sutent daily and oral MS-275 on days 3 and 10. Treatment repeats every 28 days in the absence of unacceptable toxicity. Cohorts of 3-6 patients receive escalating doses of sutent and MS-275 until the maximum tolerated dose (MTD) for the combination of sutent and MS-275 is determined. Test dose ranges are initially determined via the established individual dose ranges for MS-275 and sutent. A standard dosage for sutent is 50 mg/daily. An established dosage for MS-275 includes 2 mg/m² weekly. Additional dosages, both decreasing and increasing in amount as well a frequency, are determined based on the standard dose for both MS-275 and sutent. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive sutent as in phase I at the MTD determined in phase I and MS-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria

Blood Sampling Serial blood is drawn by direct vein puncture before and after administration of MS-275 or sutent. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon feasibility/availability, and the same imaging modality is utilized throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of MS-275 and sutent will be safe and well tolerated by renal cancer patients. The combination of MS-275 and sutent provides large clinical utility to renal cancer patients.

### Example 20

Human Clinical Trial of the Safety and/or Efficacy of MS-275/sorafenib combination therapy for renal cancer.

Objective: To compare the safety and pharmacokinetics of administered MS-275 and sorafenib in renal cancer patients.

Study Design This will be a Phase I, single-center, open-label, randomized dose escalation study followed by a Phase II study in EGFR inhibitor-resistant and EGFR inhibitor-sensitive renal cancer patients with disease that can be biopsied. Patients should not have had exposure to MS-275 or sorafenib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral sorafenib daily and oral MS-275 on days 3 and 10. Treatment repeats every 28 days in the absence of unacceptable toxicity. Cohorts of 3-6 patients receive escalating doses of sorafenib and MS-275 until the maximum tolerated dose (MTD) for the combination of sorafenib and MS-275 is determined. Test dose ranges are initially determined via the established individual dose ranges for MS-275 and sorafenib. A standard dosage for sorafenib is 400 mg taken twice PO per day. An established dosage for MS-275 includes 2 mg/m² weekly. Additional dosages, both decreasing and increasing in amount as well a frequency, are determined based on the standard dose for both MS-275 and sorafenib. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive sorafenib as in phase I at the MTD determined in phase I and MS-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria

Blood Sampling Serial blood is drawn by direct vein puncture before and after administration of MS-275 or sorafenib. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon feasibility/availability, and the same imaging modality is utilized throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of MS-275 and sorafenib will be safe and well tolerated by renal cancer patients. The combination of MS-275 and sorafenib provides large clinical utility to renal cancer patients.

### Example 21

Human Clinical Trial of the Safety and/or Efficacy of MS-275/vandetanib combination therapy for non-small cell lung cancer.

Objective: To compare the safety and pharmacokinetics of administered MS-275 and vandetanib in non-small cell lung cancer

Study Design This will be a Phase I, single-center, open-label, randomized dose escalation study followed by a Phase II study in EGFR inhibitor-resistant and EGFR inhibitor-sensitive non-small cell lung cancer cancer patients with disease that can be biopsied. Patients should not have had exposure to MS-275 or vandetanib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral vandetanib daily and oral MS-275 on days 3 and 10. Treatment repeats every 28 days in the absence of unacceptable toxicity. Cohorts of 3-6 patients receive escalating doses of vandetanib and MS-275 until the maximum tolerated dose (MTD) for the combination of vandetanib and MS-275 is determined. Test dose ranges are initially determined via the established individual dose ranges for MS-275 and vandetanib. A standard dosage for vandetanib is 300 mg/daily. An established dosage for MS-275 includes 2 mg/m² weekly. Additional dosages, both decreasing and increasing in amount as well a frequency, are determined based on the standard dose for both MS-275 and vandetanib. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive vandetanib as in phase I at the MTD determined in phase I and MS-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria

Blood Sampling Serial blood is drawn by direct vein puncture before and after administration of MS-275 or vandetanib. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon feasibility/availability, and the same imaging modality is utilized throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of MS-275 and vandetanib will be safe and well tolerated by non-small cell lung cancer patients. The combination of MS-275 and vandetanib provides large clinical utility to non-small cell lung cancer patients patients.

### Example 22

Human Clinical Trial of the Safety and/or Efficacy of MS-275/vandetanib combination therapy for colon cancer.

Objective: To compare the safety and pharmacokinetics of administered MS-275 and vandetanib in colon cancer

Study Design This will be a Phase I, single-center, open-label, randomized dose escalation study followed by a Phase II study in EGFR inhibitor-resistant and EGFR inhibitor-sensitive colon cancer patients with disease that can be biopsied. Patients should not have had exposure to MS-275 or vandetanib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral vandetanib daily and oral MS-275 on days 3 and 10. Treatment repeats every 28 days in the absence of unacceptable toxicity. Cohorts of 3-6 patients receive escalating doses of vandetanib and MS-275 until the maximum tolerated dose (MTD) for the combination of vandetanib and MS-275 is determined. Test dose ranges are initially determined via the established individual dose ranges for MS-275 and vandetanib. A standard dosage for vandetanib is 300 mg/daily. An established dosage for MS-275 includes 2 mg/m² weekly. Additional dosages, both decreasing and increasing in amount as well a frequency, are determined based on the standard dose for both MS-275 and vandetanib. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive vandetanib as in phase I at the MTD determined in phase I and MS-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria

Blood Sampling Serial blood is drawn by direct vein puncture before and after administration of MS-275 or vandetanib. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon feasibility/availability, and the same imaging modality is utilized throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype (i.e., EGFR expression, E-cadherin expression, Her2, Her3 expression) and clonogenic growth by flow cytometry, Western blotting, and IHC, and for changes in cytogenetics (i.e., EGFR, E-cadherin, and Her3 gene duplication/polysomy) by FISH. After completion of study treatment, patients are followed periodically for 4 weeks.

In conclusion, administration of a combination of MS-275 and vandetanib will be safe and well tolerated by colon cancer patients. The combination of MS-275 and vandetanib provides large clinical utility to colon cancer patients patients.

All publications and patent applications mentioned in this specification are herein incorporated by reference to the same extent as if each individual publication or patent application was specifically and individually indicated to be incorporated by reference.

Each reference cited herein is incorporated by reference in its entirety.

### REFERENCES

Jemal et al., CA Cancer J. Clin. 54(1):8-29, 2004.
Parkin, The Lancet Oncology 2:533-543, 2001.
Hirsch et al., Cancer 41 Suppl 1:S29-42, 2003.
Arteaga., Exp Cell Res 284:122-130, 2003.
Yarden and Sliwkowski, Nat Rev Mol Cell Biol. 2:127-137, 2001.
Jorissen et al., Exp Cell Res 284:31-53, 2003.
Levitzki and Gazit, Science 267:1782-8, 1995.
Fukuoka et al., J Clin Oncol. 21:2237-2246, 2003.
Kris et al., J. Am. Med. Assoc. 290, 2149-2158, 2003.
Perez-Soler et al., Proc. Am. Soc. Clin. Oncol., 20: 310a (1235) 2001.
Shepherd et al., Journal of Clinical Oncology, 2004 ASCO Annual Meeting Proceedings (Post-Meeting Edition). Vol 22, No 14S (July 15 Supplement), 2004: 7022.
Lynch et al., N Engl J Med 350:2129-39, 2004.
Paez et al., Science (Wash DC) 304:1497-500, 2004.
Pao et al., Proc Natl Acad Sci USA 101 (36):13306-11, 2004.
Cappuzzo et al., J Natl Cancer Inst 96:2004.
Reginato et al., Nat Cell Biol. 5(8):733-40, 2003.
Dumstrei et al., Development; 129(17):3983-94, 2002.
Al Moustafa et al., Lung Cancer. 37:49-56, 2002.
Qian et al., EMBO J. 23:1739-84, 2004.
Pece et al., J Biol Chem 274(27):19347-51, 1999.
Pece and Gutkind, J Biol Chem. 275(52):41227-33, 2000.
Bremnes et al., J Clin Oncol. 20:2417-2428, 2002.
Kintner, Cell 69:225-236, 1992.
Jiang, Br J Surg 83:437-446, 1996.
Ohira et al., Proc Natl Acad Sci USA. 100: 10429-10434, 2003.
Conacci-Sorrell et al., J Cell Biol. 163(4):847-57, 2003.
Lu et al., Cancer Cell. 4(6):499-515, 2003.
Batsche et al., Mol Cell Biol. 18(7):3647-58, 1998.
Bolos et al., J. Cell Sci. 116:499-511, 2003.
van Grunsven et al., J Biol Chem. 278:26135-26145, 2003.
Comijn et al., Mol Cell; 7(6):1267-78, 2001.
Verschueren et al., J Biol Chem. 274:20489-98, 1999.
Sekido et al., Mol Cell Biol. 14:5692-700, 1994.
Cano et al., Nat. Cell Biol. 2:76-83, 2000.
Hajra et al., Cancer Res. 62:1613-1618, 2002.
Chinnadurai, Mol. Cell 9, 213-224, 2002.
Postigo and Dean, Proc. Natl. Acad. Sci. USA 96, pp. 6683-6688, 1999.
de Ruijter et al., Biochem J. 370:737-749, 2003.
Marks et al., J Natl Cancer Inst (Bethesda), 92:1210-6, 2000.
Zelent et al., Clin Cancer Res 10: 4622-4629, 2004.
Gore et al., 2004 ASCO Annual Meeting Proceedings Vol 22, No 14S (July 15 Supplement): 3026, 2004.
Huelsken et al., Curr. Opin. Genet. Dev. 11, 547-553, 2001.
Cowley et al., J Pathol. 179:183-7, 1996.
Suzuki et al., Lung Cancer.; 42(1):35-41, 2003.
Cappuzzo et al., J Clin Oncol. 21(14):2658-63, 2003.
Fricke et al., Oncology 66(2):150-9, 2004.
Satoh et al., Biocell. 27(1):47-55, 2003.
Rosivatz et al., Int J Cancer 111(5):711-9, 2004.
Ozawa et al., EMBO J. 8:1711-1717, 1989.
DiGiuseppe et al., Leukemia 13:1243-1253, 1999.
Chinnaiyan et al., Journal of Clinical Oncology, 2004 ASCO Annual Meeting Proceedings (Post-Meeting Edition). Vol 22, No 14S (July 15 Supplement): 3029, 2004.

What is claimed is:

1. A method for treating cancer in a patient, comprising administering to the patient a combination of at least one selective histone deacetylase (HDAC) inhibitor and at least one kinase inhibitor with anti-EGFR activity.

2. The method of claim 1, wherein the selective HDAC inhibitor improves the sensitivity of the cancer to the kinase inhibitor.

3. The method of claim 1, wherein the selective HDAC inhibitor is MS-275 or MGCD0103.

4. The method of claim 1, wherein the kinase inhibitor is a dual kinase inhibitor.

5. The method of claim 1, wherein the kinase inhibitor is gefitinib, erlotinib, lapatinib, sutent, vandetanib, or sorafenib.

6. The method of claim 1, wherein the cancer is of epithelial origin.

7. The method of claim 1, wherein the cancer is selected from breast cancer, skin cancer, bladder cancer, colon cancer, prostate cancer, uterine cancer, cervical cancer, ovarian cancer, esophageal cancer, stomach cancer, gastrointestinal cancer, pancreatic cancer, laryngeal cancer, and lung cancer.

8. The method of claim 1, wherein the selective HDAC inhibitor is administered before the kinase inhibitor.

9. A method for treating cancer in a patient, comprising administering to the patient a combination of at least one selective histone deacetylase (HDAC) inhibitor and lapatinib.

10. The method of claim 9, wherein the selective HDAC inhibitor is MS-275.

11. The method of claim 9, wherein the cancer is selected from among breast cancer, skin cancer, bladder cancer, colon cancer, prostate cancer, uterine cancer, cervical cancer, ovarian cancer, esophageal cancer, stomach cancer, gastrointestinal cancer (GI), pancreatic cancer, laryngeal cancer, and lung cancer.

12. A method for treating cancer in a patient, comprising administering to the patient a combination of MS-275 and at least one kinase inhibitor with anti-EGFR activity.
